(19) United States
(12) Patent Application Publication
(10) Pub. No.: US 2026/0027536 A1
(43) Pub. Date: Jan. 29, 2026

(54) PROCESS FOR CONTINUOUS CATALYTIC HYDROGENATION OF MDA

(71) Applicant: Evonik Operations GmbH, Essen (DE)

(72) Inventors: Hanns Kuhlmann, Sprockhoevel (DE); Armin Matthias Rix, Marl (DE); Niklas Paul, Marl (DE); Lea Wessner, Dortmund (DE); Maria Vargas Gómez, Haltern am See (DE); Tobias Winkler, Duelmen (DE); Florian Boeck, Witten (DE); Daniel Sudhoff, Bergkamen (DE)

(73) Assignee: Evonik Operations GmbH, Essen (DE)

(21) Appl. No.: 19/276,351

(22) Filed: Jul. 22, 2025

(30) Foreign Application Priority Data
Jul. 26, 2024 (EP) 24191101.5

(51) Int. Cl. B01J 8/02; B01J 4/00; B01J 8/04; C07C 209/72

(52) U.S. Cl. CPC B01J 8/0285; B01J 4/001; B01J 8/0278; B01J 8/0492; B01J 8/0496; C07C 209/72; B01J 2204/002; B01J 2204/007; B01J 2208/00115; B01J 2208/00176; C07C 2601/14

(57) ABSTRACT

A plant for continuous catalytic hydrogenation of methylenedianiline (MDA; reactant1) with a hydrogen donor (reactant2), especially a gaseous hydrogen donor, preferably hydrogen (H2), including a conditioning unit for the reactants, a reactor unit for synthesis of PACM, and a separation unit, wherein the conditioning unit includes at least part of the length of the (feed) conduits for reactant1, reactant2 and at least one solvent, at least one heat exchanger in at least one (feed) conduit, at least one mixer for mixing the reactants and/or at least one reactant with at least one solvent; the reactor unit includes at least one fixed bed reactor as main reactor with an immobile catalyst packing.

### CROSS-REFERENCE TO RELATED APPLICATIONS

This application is a New U.S. Patent Application which claims priority to European Patent Application No. EP 24191101.5, filed on Jul. 26, 2024, the content of which is hereby incorporated by reference in its entirety.

### BACKGROUND OF THE INVENTION

### Field of the Invention

The invention relates to a plant and to a process for continuous catalytic hydrogenation of MDA, especially for production of methylenebis(cyclohexylamine), such as 4,4′-diaminodicyclohexylmethane (PACM) in particular.

Processes for hydrogenation of organic compounds, especially for hydrogenation of aromatic compounds to the corresponding cyclohexane derivatives, are already known from the related art.

Methylenebis(cyclohexylamine) is a cycloaliphatic amine that is solid or liquid under standard conditions (SATP) and is typically produced by liquid phase hydrogenation of MDA. The acronym MDA was introduced historically as an abbreviation for the product mixture which is formed in the reaction of aniline and formaldehyde and comprises mainly “methylenedianiline” (diaminodiphenylmethane), and is still used to describe the process product, which is now produced on an industrial scale. The hydrogenation product, comprising mainly methylenebis(cyclohexylamine), is therefore often also referred to as H12MDA.

Because of the process for production thereof, MDA is typically a mixture of different diaminodiphenylmethanes. It consists mainly of 4,4′-diaminodiphenylmethane. However, 2,4′ and 2,2′ isomers may also be present. MDA may also contain reaction products having three or more aromatic rings that are formed in the reaction of aniline and formaldehyde, in particular those having three or more phenyl rings. These reaction products having three or more aromatic rings are also referred to as polynuclear compounds.

Because of the high proportion of 4,4′-diaminodiphenylmethane in the MDA used, commercially available methylenebis(cyclohexylamine) is mostly 4,4′-diaminodicyclohexylmethane or bis(para-aminocyclohexyl)methane. Because of the potential presence of the corresponding 2,4′-and 2,2′-diaminophenylmethane isomers in the MDA, 2,4′-diaminodicyclohexylmethane and 2,2′-diaminodicyclohexylmethane may also be present in methylenebis(cyclohexylamine). In addition, hydrogenated MDA may also contain (optionally partly) hydrogenated polynuclear compounds as well as methylenebis(cyclohexylamine).

### Description of Related Art

U.S. Pat. No. 5,578,546 A discloses that a process for producing methylenebis(cyclohexylamine) was first described in 1947 and converted to industrial scale in 1965. Hydrogenation of MDA is highly exothermic. For example, WO 2010/069484 A1 indicates an enthalpy of reaction of −1600 KJ/mol.

As a result of hydrogenation, various diastereoisomers are formed depending on the process. The 4,4′-diaminodicyclohexylmethane (PACM) product derived from 4,4′-diaminodiphenylmethane may take the form of trans/trans, cis/cis and cis/trans isomers and is therefore generally a mixture of these isomers with different proportions. The melting point of the compound rises with rising trans/trans content. Therefore, the fields of application vary significantly depending on the isomer content: While methylenebis(cyclohexylamine) qualities with a low trans/trans content (e.g. 10-30% by weight) are used in the field of amine and isocyanate crosslinkers, especially in the field of two-component resins, qualities with a high trans/trans content (e.g. ≥48% by weight) are mainly used as regulator in polyamide compounds. The production of products with a low trans/trans content in particular is a challenge, since the thermodynamic equilibrium, as described in U.S. Pat. No. 3,636,108 A, is in the range of significantly higher trans/trans contents (up to 51.2%). In addition, U.S. Pat. No. 2,606,925 A shows that the equilibrium can be shifted retrospectively by prolonged heat treatment in the direction of a higher proportion of trans/trans isomer.

The composition of the hydrogenation product also depends on the composition of the MDA used: MDA is often used in qualities from MDA50 to MDA100, with the number between 50 and 100 indicating the content of diaminodiphenylmethanes in the MDA mixture. MDA50 is an MDA quality which, as explained above, for process-related reasons, contains about 50% by weight of diaminodiphenylmethanes and 50% by weight of polynuclear compounds. The individual polynuclear compounds can be referred to as trinuclear compounds, tetranuclear compounds, etc. according to the number of aromatic nuclei present. MDA50 is the quality produced in the highest volume and is mainly processed to methylene dicyclohexyl diisocyanate (MDI). MDA100 is pure MDA or diaminodiphenylmethane without polynuclear compounds. MDA85 or MDA90 are other grades of medium purity available on the market. When patent specifications relating to the process for production of methylenebis(cyclohexylamine) address the purity of the MDA quality, the quality in question is usually MDA100 (e.g. CN 110204447 B). US 2005/261525 A1, by contrast, prefers the hydrogenation of MDA50. The hydrogenated oligomeric amines obtained here as high boilers are suitable as crosslinkers having particularly low vapour pressures for a range of specialty applications, as shown by US 2004/162409 A1.

The content of (possibly partly) hydrogenated polynuclear compounds in the product decreases in the order of the reactants MDA50, MDA85, MDA90, MDA100, since the content of polynuclear compounds decreases from MDA50 to MDA100.

WO 2009/153123 A1 discloses a continuous process and a reactor for hydrogenation of organic compounds in a polyphasic multistage system in the presence of a homogeneous or heterogeneous catalyst. Catalysts proposed include precious metals such as platinum, palladium, ruthenium and rhodium, or other transition metals such as molybdenum, tungsten and chromium. It is possible here for the heterogeneous catalysts to be disposed on support materials, for example carbon, aluminium oxide, silicon dioxide, zirconium dioxide, zeolites, aluminosilicates or mixtures of these support materials. Substrates used in this process are preferably aromatic compounds containing amino substituents, for example MDA, polymer MDA, aniline, 2,4-diaminotoluene, 2,6-diaminotoluene, o-phenylenediamine, etc. The heterogeneous catalysts are used in suspension.

DE 19533718 A1 discloses a process for hydrogenation of aromatic compounds in which at least one amino group is bonded to an aromatic nucleus. For this purpose, it is possible to use a heterogeneous catalyst containing ruthenium and optionally at least one metal of transition group I, VII or VIII. The support material used is, for example, aluminium oxide, silicon dioxide, titanium dioxide or zirconium dioxide, preferably aluminium dioxide or zirconium dioxide. Only a catalyst containing ruthenium on the support material aluminium oxide is given as an example, and not zirconium oxide.

EP 1337331 A1 discloses a process for catalytic hydrogenation of aromatic or heteroaromatic amines, wherein ruthenium acts as the active metal and the catalyst contains at least one additional metal of transition group I, VII, or VIII, and these are applied to a support material. Aromatic compounds used here include 4,4′-MDA and isomers thereof. EP 0111238 A1 also discloses a process for catalytic hydrogenation of 4,4′-MDA, characterized in that the hydrogenation is effected in the presence of supported ruthenium in the presence of nitrates and sulfates of the alkali metals and nitrates of the alkaline earth metals. A comparable process is disclosed in EP 1366812 A1, where support materials mentioned include aluminium oxide, silicon oxide, titanium oxide and zirconium oxide.

DE 101 19 135 A1 discloses a plant and a process for production of diaminodicyclohexylmethane (PACM) with a proportion of trans, trans-4,4′-diaminodicyclohexylmethane of 17% to 24%, where this is achieved by hydrogenation of diaminodiphenylmethane (MDA) in the presence of a pulverulent catalyst. The hydrogenation is conducted here in a continuously operated suspension reactor, in particular in a cascade of multiple series-connected suspension reactors, such that an MDA conversion of at least 95% is achieved, based on the amount of MDA used.

EP 1 519 912 B1 discloses a plant and a process for production of 4,4′-diaminodicyclohexylmethane (4,4′-HMDA), wherein by means of catalytic hydrogenation of a substance mixture comprising 4,4′-diaminodiphenylmethane (4,4′-MDA) as the main component and its mono-N-methyl derivative as a secondary component with elevated selectivity with regard to the hydrogenation of 4,4′-MDA in the presence of a heterogeneous hydrogenation catalyst at a temperature in the range from 50 to 220° C. and a hydrogen pressure in the range from 1 to 30 MPa is effected. For this purpose, EP 1 519 912 B1 suggest sending the hydrogenation prior to attainment of a conversion of 4,4′-MDA to 4,4′-HMDA of 99%, especially in the range from 90% to 98.9%.

Further processes for hydrogenation of organic compounds are disclosed by WO 2011/003899 A1 and WO 2009/090179 A1. What are disclosed here are processes for hydrogenation of aromatic amines with hydrogen in the presence of an Ru catalyst containing zirconium oxide support material inter alia.

Finally, EP 2 883 863 B1 (D1b) discloses a process and a plant for hydrogenation of 4,4′-methylenedianiline (MDA) and/or polymer MDA with hydrogen in the presence of a catalyst. A catalyst proposed here is ruthenium applied to a zirconium oxide support material. With regard to the reactor, EP 2 883 863 B1 refers to the reactor or the reactor concept of WO 2008/015135 A1 (D1a). Said WO 2008/015135 A1 discloses a continuous process and a plant for hydrogenation of diisononyl phthalate to diisononyl cyclohexane-1,2-dicarboxylate (DINCH), wherein DINP is hydrogenated as a mixture in an organic solvent with hydrogen at a pressure of up to 325 bar. What is proposed here is series connection of two fixed bed reactors each with an immobile fixed bed. In order to dissipate the heat of reaction from the two reactors, it is proposed that a substream of the stream of matter be recirculated and thereby cooled downstream of the second fixed bed reactor. A comparable reactor concept of series-connected fixed bed reactors for hydrogenation of is also known from EP 1 566 372 B1.

This concept is disadvantageous in that the circulation of a product substream can lead to increased formation of unwanted by-products and reduces plant performance, causing increased energy costs for the recycling and cooling of the recycling stream.

As explained, the need for PACM, for example, with different proportions of the respective isomers is dependent on the intended use or the subsequent products. For example, PACM qualities with a low trans/trans content of 10% to 30% by weight are thus preferred in the field of amine and isocyanate crosslinkers, especially in the field of formulation of 2-component resins, and PACM qualities with a high trans/trans content exceeding 48% by weight are mainly used as regulator in polyamide compounds. The percentages by weight mentioned are based here on the PACM isomer mixture as such. The production of products with a low trans/trans content in particular is a process engineering challenge, since the thermodynamic equilibrium, as described in U.S. Pat. No. 3,636,108 A, is in the range of significantly higher trans/trans contents of up to 51.2% by weight. Furthermore, it is known from U.S. Pat. No. 2,606,925 A that the equilibrium of the PACM isomers is subsequently shifted in the direction of a higher proportion of trans/trans isomer by prolonged heat treatment.

It is thus an object of the present invention to provide an improved plant and an improved process which is improved in terms of product conversion and energy efficiency and in particular enables the production of defined proportions of the respective isomers in the isomer mixture.

The object is achieved in accordance with the invention by a plant according to the features of Embodiment 1 and a process according to the features of Embodiment 14.

### SUMMARY OF THE INVENTION

What is provided here is a plant for continuous catalytic hydrogenation of methylenedianiline (MDA; reactant1) with a hydrogen donor (reactant2), especially a gaseous hydrogen donor, preferably hydrogen (H2),
comprising a conditioning unit (104) for the reactants, a reactor unit (102) and a separation unit (106), wherein
the conditioning unit comprises (feed) conduits for reactants1, reactant2 and at least one solvent, at least one heat exchanger in at least one (feed) conduit, at least one mixer for mixing the reactants and/or at least one reactant with at least one solvent,
the reactor unit comprises at least one fixed bed reactor as main reactor with an immobile catalyst packing, wherein the at least one main reactor comprises
   a first flow pathway for the mixture of matter through the immobile catalyst packing and
   another separate closed flow pathway for a heat exchange medium outside the catalyst packing, and
   wherein the media circuit incorporates a heat exchanger;
the separation unit comprises at least
a first separation stage for removal of the solvent and
a second separation stage for separation of the at least one reactant and/or at least one by-product from the product, where
   i) a collection circuit for an open or a closed first media circuit is included, in which incorporated heat source(s) are at least the heat exchanger of the main reactor and/or at least one heat exchanger of the separation unit, and an evaporator,
   ii) an intermediate circuit for a second media circuit is included, incorporating the first evaporator, at least one fan and a distributor tank, where an incoming (media) conduit leads from the pressure side of the at least one fan to the distributor tank and a (bottom) outlet of the distributor tank is connected to an inlet of the evaporator via a return conduit, and where
   iii) at least one distributor circuit incorporating the distributor tank is included, where the distributor circuit is connected by at least one incoming conduit branch to a tops/vapour outlet of the distributor tank, where the distributor circuit comprises at least one distributor conduit branch and at least one return conduit branch, where a heat sink incorporated into the at least one distributor conduit branch so as to exchange heat is at least one heat exchanger of the conditioning unit, the separation unit, and/or the reaction unit, and where the intermediate circuit and the distributor circuit are connected to one another so as to conduct media via the distributor tank.

In this context,
the collection circuit comprises a conduit branch leading to the first evaporator or collecting energy, and a return conduit branch,
the intermediate circuit comprises a conduit branch leading to the distributor tank, also called vapour conduit, and a return conduit branch from the distributor tank to the first evaporator, also called return conduit, and
the distributor circuit comprises at least one incoming conduit branch, also called conduit section, from the distributor tank to the at least one heat exchanger of the conditioning unit, separation unit and/or reactor unit which is incorporated as a heat sink, and additionally a return conduit branch from the at least one heat exchanger acting as a heat sink, especially the group of at least two heat exchangers, to the distributor tank.

The invention includes but is not limited to the following embodiments:
1. A plant for continuous catalytic hydrogenation of methylenedianiline (MDA; reactant1) with a hydrogen donor (reactant2),
comprising a conditioning unit for the reactants, a reactor unit and a separation unit, wherein
the conditioning unit comprises (feed) conduits for reactant1, reactant2 and at least one solvent, at least one heat exchanger in at least one (feed) conduit, at least one mixer for mixing the reactants and/or at least one reactant with at least one solvent; the reactor unit comprises at least one fixed bed reactor as main reactor with an immobile catalyst packing, wherein the at least one (first) main reactor comprises a first flow pathway for the mixture of matter through the immobile catalyst packing and another separate closed flow pathway for a heat exchange medium outside the catalyst packing, and
wherein the media circuit incorporates a heat exchanger;
the separation unit comprises at least
a first separation stage for essentially removal of the solvent and
a second separation stage for separation of the at least one reactant and/or at least one by-product from the product,
wherein
i) a collection circuit for an open or a closed first media circuit is included, in which at least one heat source incorporated is a heat exchanger of the reactor unit, and/or at least one heat exchanger of the separation unit and an evaporator or a distributor tank,
ii) an intermediate circuit for a second media circuit is included, incorporating the first evaporator, at least one fan and a distributor tank, where a (media) conduit leads from the pressure side of the at least one compressor to the distributor tank and a (bottom) outlet of the distributor tank is connected to an inlet of the evaporator via a return conduit, and where
iii) at least one distributor circuit incorporating the distributor tank is included, where the distributor circuit is connected by at least one incoming conduit branch to a tops/vapour outlet of the distributor tank, where the distributor circuit comprises at least one distributor conduit branch and at least one return conduit branch, where a heat sink incorporated into the at least one distributor conduit branch so as to exchange heat is at least one heat exchanger of the conditioning unit, the separation unit, and/or the reaction unit, and where
the intermediate circuit and the distributor circuit are connected to one another via the distributor tank so as to conduct media.

2. The plant according to Embodiment 1, wherein the at least one incoming conduit branch of the distributor circuit incorporates at least one compressor.

3. The plant according to Embodiment 1, wherein the distributor circuit comprises at least the following two (part-)circuits:
at least one high-pressure distributor circuit (HP distributor circuit) connected to a tops/vapour outlet of the distributor tank and incorporating at least one compressor, and at least one low-pressure distributor circuit (LP distributor circuit) connected to a tops/vapour outlet of the distributor tank and incorporating no compressor, fewer compressors and/or a lower compressor output than the HP distributor circuit, and where the HP distributor circuit and the LP distributor circuit incorporate, so as to exchange heat,
as a heat sink at least one heat exchanger of the conditioning unit, separation unit, and/or of the reaction unit, and where the intermediate circuit and the distributor circuit are connected to one another via the distributor tank so as to conduct media.

4. The plant according to Embodiment 1, wherein the intermediate circuit comprises at least one fan or a group of x fans where x is >=2.

5. The plant according to Embodiment 1, wherein the media circuit of the collection circuit incorporates a collection tank and a pump, where a heat exchanger is disposed upstream of the collection tank in the return conduit branch and/or on the suction side of the pump in the incoming conduit branch.

6. The plant according to Embodiment 3, wherein the HP distributor circuit of the distributor circuit comprises a first conduit branch as a vapour-conducting feed, where the feed conduit branch incorporates at least one compressor.

7. The plant according to Embodiment 3, wherein a (bottoms) conduit leads from the distributor tank to the pressure side of the at least one compressor, where this (bottoms) conduit incorporates a pump.

8. The plant according to Embodiment 7, wherein the bottoms conduit from the distributor tank is guided into the (media) conduit between two compressors.

9. The plant according to Embodiment 7, wherein the bottoms conduit has at least two (conduit) branches, where each (conduit) branch of the bottoms conduit is guided between two of the compressors, and where at least one (conduit) branch of the bottoms conduit comprises a pressure control unit.

10. The plant according to Embodiment 3, wherein LP distributor circuit of the distributor circuit comprises at least two part-circuits for distributing or releasing energy, where the first part-circuit is a low-pressure circuit (LP part-circuit), where the first LP part-circuit incorporates at least one heat exchanger or a first group of two or more heat exchangers, and where the further part-circuit incorporates at least one heat exchanger or a further group of two or more heat exchangers, with no compressor, fewer compressors and/or a lower compressor output incorporated into the LP part-circuit than in any other part-circuit of the LP distributor circuit.

11. The plant according to Embodiment 10, wherein at least one of the return conduits, returning to the distributor tank, of the distributor circuit, incorporates a heat exchanger.

12. The plant according to Embodiment 1, wherein the heat exchangers of the distributor circuit that act as a heat sink are connected in parallel and/or the respective groups of two or more heat exchangers of the HP distributor circuit and/or the LP distributor circuit are connected in parallel to one other.

13. The plant according to Embodiment 1, wherein the heat exchangers, acting as heat source, of the collection circuit are connected in series.

14. A process for catalytic hydrogenation of methylenedianiline (MDA; reactant1) with a hydrogen donor (reactant2), the process comprises:
implementing a production by an industrial plant,
wherein
the plant is designed according to Embodiment 1, where the main reactor is operated at a temperature in the range from 80° C. to 150° C., and where vapour compression in the intermediate circuit by the at least one fan and/or the fan group results in at least an increase in temperature of the medium in the incoming conduit branch by 20° C. to 60° C.

15. The process according to Embodiment 14, wherein the temperature of the reactant stream at the inlet of the at least one main reactor is 80 to 135° C.

16. The process according to Embodiment 14, wherein the pressure in the main reactor is operated in the range from 60 bar to 120 bar.

17. The process according to Embodiment 14, wherein it is implemented continuously and catalytically for production of methylenebis(cyclohexylamine).

18. The process according to Embodiment 14, wherein in addition to the at least one main reactor, at least one catalyst-containing postreactor is also included, where the at least one main reactor and the at least one postreactor are operated at the same or essentially at the same pressure.

19. The process according to Embodiment 14, wherein the energy introduced into the first evaporator by the collection circuit by the intermediate circuit and the at least one incorporated fan and/or the group of x fans is raised
by at least a factor of 1.1 to 2.5 and/or
the output temperature of the tank is raised by 10° C. to 80° C.

20. The process according to Embodiment 14, wherein a multistage pressure elevation in the incoming conduit is effected in the first conduit portion (portion A) in the HP distributor circuit, where, downstream of the distributor tank and
upstream of the first compressor in the conduit, an (input) pressure is 1.5 bar to 5 bar, and a temperature is 100° C. to 150° C. and/or downstream of the last compressor and upstream of the first heat exchanger acting as a heat sink, a (final) pressure in the conduit is 10 to 30 bar and a temperature is 180° C. to 300° C.

### BRIEF DESCRIPTION OF THE DRAWINGS

FIG. 1 shows a plant as a process flow diagram.

FIG. 2 shows a first embodiment of the three media circuits.

FIG. 3 shows a second embodiment of the media circuits with an option for direct interconnection of a group of heat exchangers.

FIG. 4 shows a third embodiment of the media circuits with an option for direct interconnection of a group of heat exchangers.

FIG. 5 shows one embodiment of the reactor unit.

FIG. 6 shows a further embodiment of the reactor unit.

FIG. 7 shows a fourth embodiment of the media circuits with a further distributor tank between collection circuit and intermediate circuit.

FIG. 8 shows a fifth embodiment of the media circuits, as a variant of the embodiment of FIG. 7.

### DETAILED DESCRIPTION OF THE INVENTION

The special feature of these three circuits is that the collection circuit is a closed media circuit or an open media circuit. What is meant in the present context by “closed (media) circuit” is that the heat exchange medium, generally water, is conducted in a closed circuit. The heat exchange from the collection circuit with the intermediate circuit is effected in indirect heat transfer via the evaporator. In a closed circuit, the (heat exchange) medium can circulate only in the respective circuit, and in particular not be passed onward into the adjacent circuit, such that the exchange of energy with the respectively coupled adjacent circuit is effected via indirect heat transport between the circulating (circulation) media, without exchange of the respective (circulation) media.

The intermediate circuit here is coupled to the collection circuit and the at least one distributor circuit. The collection circuit serves as heat source for the intermediate circuit; the intermediate circuit serves to raise the temperature level and as a heat source for the distributor circuit and the heat exchangers incorporated therein that function as a heat sink, especially the heat exchangers of the separation unit.

In contrast, what is meant in the present context by “open (media) circuit” is that the heat exchange medium, generally water, is directed from one circuit at least in part or at least in one state of matter into an adjacent circuit, or withdrawn or received therefrom. Media exchange in the case of open (media) circuits is effected via distributor tanks in particular.

Thus, in the present context, the term “evaporator” means that indirect heat transfer is effected and no exchange of matter takes place between incoming substances or media. In contrast, what is meant in the present context by “distributor tank” is that at least portions of media flowing in from two or more (part-)circuits are physically mixed, i.e. coalesce. In this case, a distributor tank may additionally comprise or be connected to a heat exchange element by means of which indirect heat transfer can take place.

The media circuit of the intermediate circuit and of the distributor circuit are fluidically coupled via the distributor tank such that the same heat exchange medium, generally water or water vapour, flows through the two circuits. The distributor tank forms a kind of common flow node or conduit node for the intermediate circuit and the distributor circuit.

The “distributor tank” should also not be comprehended in a restricted manner in the present context and represents an arbitrarily shaped tank or space in which a phase separation into vapour phase and liquid phase can be effected, such that the liquid phase can be conveyed separately back to the evaporator, for example from the bottom of a tank. Thus, the distributor tank has a bottoms outlet to which a bottoms conduit is connected and at least one (bottoms) pump is connected. The (bottoms) pump charges at least the return conduit of the intermediate circuit with (heat exchange) medium to the evaporator, where it is again at least partly evaporated.

The liquid stream of matter coming from the separation tank is directed via a conduit to the first separation column within the first separation stage of the separation unit. Advantageously, in one embodiment of the plant, it may be the case that a pressure control unit for lowering pressure is provided in the conduit from the separating tank to the first column. This allows the reactor unit to be operated at a first, high pressure level, and the first separation stage of the separation unit at a second, lower pressure level.

The plant is preferably used for continuous catalytic production of methylenebis(cyclohexylamine) as product, especially for production of 4,4′-diaminodicyclohexylmethane (PACM), of the formula (I)

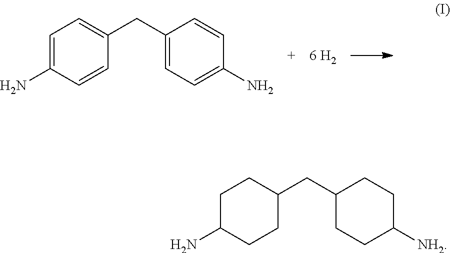 [SMILES: Nc1ccc(Cc2ccc(N)cc2)cc1 ; NC1CCC(CC2CCC(N)CC2)CC1]

With the separate postreactor, especially the adiabatic postreactor, an optimized control of the process and the plant has been enabled, whereby selectivity becomes possible because of the variance of the inlet temperature of the postreactor from the main reactor (outlet). Typically, the inlet temperature of the postreactor may be provided at the same or a slightly lower temperature, which may be up to 30° C. below the outlet temperature of the main reactor. Thus, at least temporarily, an increase in temperature in the (adiabatic) postreactor of 20° C. to 40° C., typically of 20° C. to 30° C., may be allowed, and hence the desired product quality (isomer ratio) can be specifically adjusted. In this way, a very low and a very exact proportion of trans/trans isomers in the isomer mixture can be established. The main reactor can be operated at a very low temperature level such that the trans/trans content of the PACM in the stream of matter (outlet of the main reactor) is about 13% to 20% by weight, with about 13% being achievable in the case of a new or regenerated catalyst and 20% by weight in the case of a catalyst after prolonged use (shortly before replacement/regeneration). Depending on the main reactor phase, it may be useful at least temporarily if the inlet temperature of the postreactor is 5 to 20° C. higher than that of the main reactor.

The main reactor that has been freshly filled with catalyst or regenerated, with the strongly exothermic reaction therein, is operated largely isothermally, although this should not be understood in the ideal sense. Even though the main reactor is referred to in the present context as being “isothermal”, this ideal state is only achieved to a limited degree in industrial use, and so a temperature gradient of about 5 to 10° C. in radial direction and also in flow direction develops within the main reactor owing to incomplete heat dissipation.

The postreactor is charged via the upstream heat exchanger with a somewhat higher temperature so as to result in the desired residual reaction and isomerization to give the final desired trans/trans ratio of 17% to 23% by weight, for example. The postreactor with the weakly exothermic residual reaction therein is operated in a largely adiabatic manner, although this should not be understood in the ideal sense.

As time advances, the catalyst activity decreases until replacement or regeneration is required. For this purpose, in parallel, the operating temperature is raised by controlling the cooling circuit, i.e. by cooling to a lesser degree via the heat exchanger incorporated in the cooling circuit in order to keep conversion and selectivity at a largely constant level overall. As a result, the isomer ratio is shifted, toward higher trans/trans contents in the product. It has been found here to be very advantageous that the stream of matter in the feed of the postreactor can be controlled in the opposite sense and autonomously by means of the upstream heat exchanger, in particular in the same sense. Thus, as the operating temperature of the main reactor rises over the service life of the catalyst, the feed temperature in the stream of matter of the postreactor is lowered.

From time t₀, the start of the process after renewal or regeneration of the catalyst, to time t₄, the end of the process determined by renewal or regeneration of the catalyst, the operating temperature of the main reactor can be increased and the temperature in the stream of matter in the inlet (feed) to the postreactor can be maintained or lowered. Advantageously, the plant is designed such that the temperature can be increased or lowered continuously and/or stepwise.

The main reactor is operated here isothermally or largely isothermally, and the postreactor adiabatically or largely adiabatically. At time t₀ (start of process), the catalyst in the postreactor may also have been renewed or regenerated as well as the main reactor. Advantageously, the cycle for regeneration of the postreactor is determined autonomously and independently of the main reactor, especially when the abovementioned interaction of the two reactors no longer enables the production of the desired low trans/trans content in the PACM.

Advantageously, the mixing unit is formed in two parts and comprises, for example, a mixing apparatus and a gas saturator. The mixing apparatus may especially be a dynamic or static mixer suitable for intimate association of MDA (reactant1) with the solvent. The gas saturator may especially be a small column or a tank, in which suitable internals are available for intensive dissolution of the H2 gas supplied under a high pressure in the MDA solvent stream of matter and/or for homogeneous distribution thereof prior to entry into the reactor. The H2 gas pressure is advantageously 70 bar to 100 bar.

The term “immobile catalyst packing” or “immobile catalyst” refers to any form of a local catalyst that does not flow or move with the stream of matter, such as, in particular, catalyst beds (pellets or coated carrier bodies) or fixed internals coated with catalyst material. Advantageous internals having a catalyst coating may be, for example, grids, plates or other bodies arranged in the main reactor.

In the present context, an “XY unit” and/or “XY stage” always also means at least one corresponding apparatus, device or the like which is included in the respective unit or stage. This means, for example, a mixing unit/stage comprising this at least one mixer/mixing device.

What is meant in the present context by “heat exchange” or “heat exchanger” is always indirect heat exchange and corresponding designs with closed material and media conduits, in the absence of any explicit description to the contrary.

In the present context, essentially the following energy couplings (EC) are considered, such as integrated energy coupling or direct energy coupling, where integrated EC means integrated energy coupling, subdivided into
a. integrated material-based energy coupling (isEC) of at least two streams of matter in a heat exchanger in indirect heat exchange, i.e. structural integration in a single heat exchanger (apparatus), meaning that, where two heat exchangers were formerly provided, both heat transfer functions of a structural unit (heat exchanger) are integrated,
b. integrated media-based energy coupling (integrated media-based EC) of at least two heat exchange media in a heat exchanger in indirect heat exchange, meaning structural integration in a single heat exchanger (apparatus).

The aforementioned ECs may be designed as direct energy coupling (direct EC) in that a serial EC or serial interconnection of at least two heat exchangers is provided.

The “condensation unit” of the first separation stage refers to a single heat exchanger or a group of heat exchangers which are used for at least partial condensation and/or cooling of the fractions of light boilers derived via the top conduit(s). A “condensation unit” referred to as such need not be a (closed) structural unit. Thus, to some degree, the term “condensation unit” and a single “heat exchanger” are also used synonymously.

The stage referred to as “first separation stage” is determined in particular in that, and has corresponding apparatuses and conduits such that, the solvent is (specifically) separated from the product-rich stream of matter and is advantageously also returned to use in the reactor unit and/or the conditioning unit. In an analogous manner, the stage of the separation unit referred to as the “second separation stage” means that it is thus determined, and has corresponding apparatuses and conduits, in order to separate the product from by-products and reactants, in particular MDA, and purify the product. In this case, the first and second stages are naturally not strictly separated, and there may be an overlap region or an apparatus in the transition region in which both solvent and at least one by-product or at least one reactant is separated from the product. In the present context, what is meant by the “removal of solvent” in the first separation stage and “separation of at least one reactant and/or at least one by-product from the product”, where the product may especially be PACM, is that the removal/separation does not mean an absolute delimitation of the separation stages, but affects in each case “essentially” only the substance(s) mentioned.

The liquid stream of matter coming from a flash separation tank, referred to hereinafter as separation tank (also called flash vessel in some cases), is directed via a conduit to the first separation column within the first separation stage of the separation unit. It is a feature of the separation tank that the incoming stream of matter is separated into a vapour phase (solvent, solvent-rich) and a liquid phase (solvent-depleted) by expansion (lowering the pressure) and that both phases are present in the separation tank in regular operation. The separation tank may additionally have a bottoms circulation system with integrated heat exchanger or be connected thereto in order to increase the removable vapour content beyond the pressure-related fraction by heating the liquid phase. Furthermore, a separating tank may comprise internals or random packings, in order in particular to prevent entrainment of droplets that have only been incompletely depleted of solvent, if at all. Advantageously, in one embodiment of the plant, it may be the case that a pressure control unit is provided in the conduit from the separating tank to the first column. This allows the reactor unit to be operated at a first, high pressure level, and the first separation stage of the separation unit at a second, lower pressure level. What is thus meant in the present context by separation tank (flash vessel) is a device whereby a phase separation is caused essentially by expansion. What is meant here by separation column, by contrast, is an apparatus in which a separation into a vapour phase and liquid phase takes place essentially by supply of energy, in particular by incorporating a tops circulation system in which at least a portion of the liquid condensed out is directed back into the column at the top.

What is meant in the present context by the term “reactant mixture” is the mixture of matter present on entry into the (first) main reactor, i.e. the mixture of all reactants, solvents, auxiliaries etc. in and downstream of the at least one reactor, the flowing mixture of matter in any degree of reaction or subsequent purification is referred to as “stream of matter” or “mixture of matter”, in some cases with addition of adjectival descriptions such as “product-rich” or “solvent-rich”. However, the physical composition of the stream of matter at each site in the plant is also obvious to the skilled person by virtue of that site in the plant and upstream plant components, and in particular the process engineering apparatuses of the plants. The pure substances, for example the product, in particular the PACM product, and the LB and HB by-products, are named and identified separately. “LB” here stands for “low boilers”, a valuable mixture of matter that is separated separately from the stream of matter and from the product and has a low boiling point of about 240° C. to 290° C. Analogously, “HB” stands for “high boilers”, a valuable mixture of matter that is separated separately from the stream of matter and from the product and has a high boiling point of >350° C.

What are describedin particular in the present context are a plant and a process for production of methylenebis(cyclohexylamine) as production 4,4′-product, especially for of diaminodicyclohexylmethane (PACM), by catalytic hydrogenation of methylenedianiline. The primary product here is PACM, which is hydrogenated from 4,4′-diaminodiphenylmethane (4,4′-MDA; primary fraction of reactant1), especially with the low trans/trans isomer ratio mentioned, and so the plant and the process and are suitable, in serve, particular for production of 4,4′-diaminodicyclohexylmethane (PACM) by continuous catalytic hydrogenation of 4,4′-diaminodiphenylmethane (4,4′-MDA). The additional fractions of 2,4′-MDA and 2,2′-MDA of the MDA present are converted at least in small proportions in parallel to reaction products, where these generally remain in the product mixture. In addition, other by-products can advantageously likewise be purified and separated off by this process or this plant in the second separation stage of the separation unit, especially in at least one separation column. These are regularly secondary (valuable) products, herein described as and meaning essentially high boilers (HB) and low boilers (LB).

The solvent is advantageously from the following group of substances: cyclohexane, dioxane, tetrahydrofuran (THF), cyclohexylamine, dicyclohexylamine, methanol, ethanol, isopropanol, n-butanol, 2-butanol, 2-methoxy-2-methylpropane (MTBE) or methylcyclohexane or a mixture thereof. Advantageously, the solvent, especially THF, is fed in excess into the MDA, such that the ratio of the mass flow rates of solvent, especially THF, to MDA at the inlet of the main reactor is advantageously in the range from 1.0 to 8.0, especially in the range from 2 to 7.5.

In a further advantageous embodiment of the plant, it may be the case that at least one compressor is incorporated in the at least one incoming conduit branch of the distributor circuit. An improvement here is that a multistage compression is provided, meaning that a group of at least two compressors are provided in the incoming conduit branch. The compression acts on the vapour coming from the collecting tank.

Even though the compression from an initial pressure at an initial temperature, as present in the distributor tank, to a desired final pressure or final temperature can in principle also be effected in a single stage, the advantage of multistage compression is that intermediate feeds, branches at intermediate pressures and intermediate temperatures are possible. Furthermore, electrical power consumption decreases if a downstream compressor only has to compress an already precompressed volume flow, and the compressors may be of structurally smaller design than a single-stage (final) compressor.

In a further advantageous embodiment of the plant, it may be the case that the distributor circuit comprises at least two (part-)circuits:
at least one high-pressure distributor circuit (HP distributor circuit) connected to a tops outlet or vapour outlet of the distributor tank and incorporating at least one compressor, and
at least one low-pressure distributor circuit (LP distributor circuit) connected to a tops/vapour outlet of the distributor tank and incorporating no compressor, fewer compressors and/or lower compressor output than the HP distributor circuit, and where
the HP distributor circuit and the LP distributor circuit incorporate, so as to exchange heat, as a heat sink at least one heat exchanger of the conditioning unit, separation unit and/or the reaction unit, and where the intermediate circuit and the distributor circuit are connected to one another via the distributor tank so as to conduct media.

What is meant here by the LP distributor circuit in one embodiment incorporating “fewer compressors and/or lower compressor power” than in the HP distributor circuit is that the possible final pressure and hence also the final temperature (without further heat exchange) is lower than in the HP distributor circuit due to the number and/or the design of the compressors in the LP distributor circuit. In other words, a lower pressure of the vapour generated than in the HP circuit is sufficient to fulfil the heating functions in the LP distributor circuit at a relatively low temperature level.

The HP distributor circuit in the present context means the (part-)circuit of the distributor circuit in which the maximum possible (final) pressure and hence generally also the highest final temperature in the distributor circuit is generatable in the vaporous medium (within the plant), which is possible owing to the power and/or number of (especially series-connected) compressors and/or the compressor output possible therewith.

In this embodiment, the HP distributor circuit and the LP distributor circuit of the distributor circuit each comprise at least one conduit branch or section for distribution or release of energy, wherein
the LP distributor circuit incorporates a first heat exchanger or a first group of two or more heat exchangers, and where
the HP distributor circuit incorporates an additional heat exchanger or a further group of two or more heat exchangers. In this way, the first heat exchanger or the first group of two or more heat exchangers of the LP distributor circuit can be operated at a first temperature and pressure level, and the further heat exchanger or the further group of two or more heat exchangers of the HP distributor circuit can be operated at a second, higher temperature and pressure level.

In this case, in addition, at least one pressure control unit is provided in a return (collection) conduit; in particular, one pressure control unit (relaxation unit) for each pressure level is provided up to introduction into the collection tank or a central return conduit which leads into the collection tank.

Furthermore, the connection “to a tops outlet and/or vapour outlet of the distributor tank” means that this connection may be a common (central) conduit from which there are branches into the HP or LP low-pressure circuit, or both circuits individually separately have in each case connections to the distributor tank.

In a further advantageous embodiment of the plant, it may be the case that the intermediate circuit, in the incoming conduit branch, incorporates a fan or a group of at least two fans. It has been found to be advantageous to perform the desired pressure rise in multiple stages because this reduces power consumption overall since the downstream compressors have to compress an ever lower vapour volume flow rate, which means that they can be of structurally smaller design. In this case, the intermediate circuit may incorporate a group of x fans where x is an integer not less than 2, especially not less than 3. The fans are connected to one other in series such that at least two-stage vapour compression is effected in the incoming conduit branch. In general, it is not advantageous to provide more than 5 or 6 fans in series. A “fan” in this context is a flow machine for gas or vapour compression, which has a ratio TT of final pressure (pressure side, output) to inlet pressure (suction side, input) of 1.3 to 2.5. Flow machines with TT>>2.5 are referred to in the present context as “compressors”.

In a further advantageous embodiment of the plant, it may be the case that the media circuit of the collection circuit incorporates a collection tank and a pump, where a heat exchanger is disposed upstream of the collection tank and/or on the suction side of the pump. In one embodiment, the heat exchanger is integrated into the collection tank and/or the collection tank has an integrated temperature control unit. The advantage here is that overall the required temperature level and the required volume flow rate of medium can be provided in the incoming conduit branch regardless of the current energy input into the collection circuit and/or the current energy consumption in the incorporated first evaporator. The circulated heat exchange medium in the collection circuit is advantageously water or a substantially aqueous solution.

In a further advantageous embodiment, it may be the case that the distributor circuit comprises a first conduit branch as a vapour-conducting feed, where this conduit branch incorporates at least one compressor, and ideally a group of compressors comprising two to five compressors, especially comprising two or three compressors, is provided. The two or more compressors are advantageously connected in series to one another.

In this way, the temperature of the vapour coming from the distributor tank in the incoming conduit can be raised with high efficiency by 30 to 150° C., especially raised by 70 to 120° C. Overall, the distributor circuit is a water circuit, or a circuit, the heat exchange medium of which is water or an aqueous solution.

In a further advantageous embodiment of the plant, it may be the case that, with two or more evaporators in the incoming conduit branch, at least one (bottoms) conduit and/or at least one branch of a (bottoms) conduit leads from the distributor tank to the pressure side of at least one compressor, with a pump incorporated in the (bottoms) conduit.

Advantageously, in one embodiment, a (bottoms) conduit and/or at least a branch of a (bottoms) conduit is guided in each case between a pair of two compressors, in particular in each case between each pair of two serial compressors. It has surprisingly been found to be energetically advantageous overall to feed the liquid medium from the (bottoms) conduit, which is at a somewhat lower temperature level, into the incoming (vapour) conduit branch. The pressure increase by pump in the liquid medium in the (bottoms) conduit and expansion via a pressure control unit is energetically advantageous compared to direct vapour compression, and so the energy requirement or the power consumption of the compressor upstream of the feed is reduced;

furthermore, the energy requirement of the respectively downstream compressor is also reduced because the vapour cooling associated with the intermediate feed reduces the volume of the compressed vapour (from the upstream compressor) while simultaneously increasing the mass flow rate through the expanded evaporated media inflow (from the intermediate feed).

Advantageously, a controllable valve or a pressure control unit is provided for this purpose in the (bottoms) conduit or in the respective branches of the (bottoms) conduit in order to control or completely shut down the volume flows. In particular, the controllable valve or the pressure control unit ensures that the liquid medium from the distributor tank and the bottoms conduit, the pressure of which is adjustable via a pump, is evaporated and introduced in vaporous form into the respective section of the incoming conduit branch.

In a further advantageous embodiment of the plant, it may be the case that the (bottoms) conduit has at least two branches, each branch of the (bottoms) conduit being guided between two of the compressors of the incoming conduit branch of the HP distributor circuit. The pump operating in the (bottoms) conduit is advantageously disposed downstream of the distributor tank and upstream of the first branch or conduit branch.

Advantageously, pressure and/or temperature sensors are provided in at least one branch of the (bottoms) conduit.

In a further advantageous embodiment of the plant, it may be the case that at least one return conduit branch of the distributor circuit incorporates a heat exchanger so as to exchange heat. This heat exchanger is incorporated downstream of the conduit branch for distribution into the return conduit branch. This heat exchanger is an on-demand or control heat exchanger that can be used to ensure that medium is provided to the distributor tank at the required temperature level regardless of the energy consumption or the energy release in the conduit branch for distribution. This on-demand or control heat exchanger is advantageously not assigned to any other apparatus in the plant, i.e. is not intended to perform any other heat exchange function than to control the inflow to the distributor tank.

In a further advantageous embodiment of the plant, it may be the case that the LP distributor circuit of the distributor circuit comprises at least two part-circuits for distributing or releasing energy, wherein
the first part-circuit is a low-pressure circuit (LP part-circuit), where the first LP part-circuit incorporates at least one heat exchanger or a first group of two or more heat exchangers, and where
the further part-circuit incorporates at least one heat exchanger or a further group of two or more heat exchangers, with no compressor, fewer compressors and/or a lower compressor output incorporated into the LP part-circuit than in any other part-circuit of the LP distributor circuit.

Each part-circuit here has a feed conduit. The respective feed conduit may in each case have autonomous compressors or compressor stages. Advantageously, the respective feed conduit for a part-circuit of the LP distributor circuit branches off at a different compressor stage from the feed conduit of the HP distributor circuit. Thus, for example, the branch to the feed conduit of the LP part-circuit may be at the lowest, first compressor stage downstream of the first compressor; the feed conduit that branches off to the second part-circuit of the LP distributor circuit branches off downstream of the second compressor of the feed conduit of the HP distributor circuit. Thus, the part-circuits have different, increasing pressure and temperature levels. This makes it possible to be able to incorporate exactly that heat exchanger or that group of heat exchangers as a heat sink for which the respective temperature level is sufficient such that energy consumption for further compression only needs to be effected in a controlled manner for the heat exchangers that require an elevated temperature level.

Analogously to the definition of the HP distributor circuit, “LP part-circuit” refers in the present case to the part-circuit operable at the lowest pressure level of all part-circuits of the LP distributor circuit because the incoming conduit, proceeding from the distributor tank, incorporates no compressor, fewer compressors and/or a lower compressor output than in any other part-circuit, this relating to regular operation of the plant. This is not supposed to mean failure or damage situations.

The part-circuits each further comprise a further conduit branch for returning the medium to the distributor tank or are connected to a central, common return conduit which is connected to the distributor tank and hence directs the medium, depleted of energy, into the distributor tank.

In an advantageous embodiment,
the HP distributor circuit incorporates at least two heat exchangers from bottom circuits of two separation columns of the separation unit, providing for three-stage compression in the incoming conduit,
the first part-circuit of the LP distributor circuit incorporates the (bottom) heat exchanger of a separation tank of the separation unit, providing for single-stage compression up to the branch of the incoming conduit, and where
the second part-circuit of the LP distributor circuit incorporates two heat exchangers from bottom circuits of two separation columns of the separation unit, providing for two-stage compression up to the branch in the incoming conduit.

Advantageously, downstream of each branch of an incoming conduit to one of the two part-circuits of the LP distributor circuit, medium from a (bottoms) conduit connected to the collecting tank is expanded and fed in intermediately.

The (sub) groups of two or more heat exchangers that are incorporated as heat sinks into one of the distributor circuits, including part-circuits, at a respective pressure level are connected in parallel to one another. The feeds to each of the heat exchangers of a group advantageously branch off from a common distributor conduit, and the outlets from each heat exchanger of a group advantageously flow into a common collection conduit. Several groups of heat exchangers may be discharged via a common collecting conduit, where the pressure levels are limited by providing at least one pressure control unit for lowering the pressure downstream of each group of heat exchangers and/or each part-circuit in the respective collecting conduit and/or the respective return conduit.

Multiple collection conduits may open into a common return conduit connected to the distributor tank.

In a further advantageous embodiment of the plant, it may be the case that the distributor circuit comprises a further conduit branch for distribution and/or release of energy and a further conduit branch for media recycling, wherein the conduit branch for distribution incorporates at least one heat exchanger of the reactor unit, at least one heat exchanger of the conditioning unit and/or at least one heat exchanger of the separation unit as a heat sink, in particular incorporates a plurality of the respective heat exchangers. In a particularly preferred variant, all other conduit branches for distributing and/or releasing energy are each connected to one branch from the incoming conduit branch, which correlate with different pressure levels and temperature levels. Thus, a first branch may be disposed downstream of the first compressor, a second branch downstream of the second compressor, etc., and/or a first branch may be disposed downstream of a first pressure control unit, a second branch downstream of a second pressure control unit, etc.

This grouping of the heat exchangers by further conduit branches allows individual heat exchangers or groups of two or more heat exchangers to be fed in a controlled manner according to energy requirement and as required by the temperature level. Thus, it is advantageously possible first to discharge a heat exchanger or a group of heat exchangers with a low temperature level and/or a high exchange power in flow direction, such that the power consumption of the downstream compressors is correspondingly reduced. As required, medium can be supplied from the (bottoms) conduit in the incoming conduit branch, especially in vaporous phase, as described above.

In a further advantageous embodiment of the plant, it may be the case that, when there is more than one return conduit branch or at least one collection conduit of the distributor circuit and/or an HP circuit, LP circuit or part-circuit, an on-demand and/or control heat exchanger is incorporated in at least one further return conduit branch so as to exchange heat, ideally with all return conduit branches incorporating such an on-demand and/or control heat exchanger. These are advantageously connected to an independent heating and/or cooling circuit and/or an independent heating and/or cooling source, such that it is possible in this way, as required, in the event of startup, maintenance and/or shutdown situations, to establish the required operating temperature in the intermediate circuit and/or distributor circuit.

In a further advantageous embodiment of the plant, it may be the case that at least some of the number of the heat exchangers in the distributor circuit incorporated as a heat sink are connected in parallel. Advantageously, the individual heat exchangers or the groups of heat exchangers connected in parallel are each controllable by open-loop and/or closed-loop control in the inlet or outlet. Controllability relates here in particular to the respective flow rates of medium, where this control is advantageously effected depending on the required temperature gradient in the respective heat exchanger or the group of heat exchangers and/or the required energy transfer. In a further advantageous embodiment, an individual heat exchanger serving as a heat sink or a group of two or more heat exchangers is not incorporated into the distributor circuit. The integration of these heat exchangers, in particular (bottom) heat exchangers of the separation columns from the separation unit, when incorporated into the distributor circuit, would have an electrical power demand for the compressors and/or another compressor which is comparable to direct electric heating of these heat exchangers. It has been found to be advantageous overall if, even in the case of an elevated energy requirement for direct electrical heating of heat exchangers of a factor of 1.2 to 1.3 compared to integration into the distributor circuit, electrical direct heating is advantageous owing to other smaller demands, such as less wear, shorter shutdown times, etc.

In a further advantageous embodiment of the plant, it may be the case that the heat exchangers, acting as a heat source, in the collection circuit are connected in series. For this purpose, the incorporated heat exchangers are advantageously incorporated into this conduit branch with a rising temperature level in flow direction of the incoming conduit branch. The central heat source used for the collection circuit is the heat exchanger incorporated in the cooling circuit of the main reactor and used to dissipate the exothermic energy from the reaction. Further heat sources used are especially the (top) heat exchangers (condensers) of the separation columns, depending on the temperature level therein in the medium of the respective heat exchanger.

The liquid stream of matter coming from the separation tank is directed via a (feed) conduit to the first separation column in the first separation stage of the separation unit. Advantageously, in one embodiment of the plant, it may be the case that a pressure control unit is provided in the (feed) conduit from the separation tank to the first column. In this way, the reactor unit may be operated at a first, high pressure level, and the first separation column of the first separation stage at a second, lower pressure level, where the separation tank may be operated at an intermediate level.

Furthermore, it may be advantageous when a heat exchanger is provided upstream of the first separation column, especially also downstream of the pressure control unit or between the pressure control unit and the first separation column.

In a particularly advantageous embodiment, an energy coupling is provided in order to operate the condenser downstream of the separation tank, a condenser of a separation column of the second separation stage or the (circulating) heat exchanger in the media circuit of the at least one main reactor interconnected in heat exchange with the (forward) heat exchanger of the first separation column. The energy coupling can be effected by conduction of media and a series interconnection of the respective heat exchangers or by integrated energy coupling in a (structurally) single heat exchanger. Integrated energy coupling has the advantage, if spatially implementable within the plant, that only a temperature gradient for heat transfer has to be overcome. It is thus possible by energy transfer to achieve a parallel rise in the (feed) stream of matter upstream of the first separation column of the first separation stage, which is at a temperature level of about 85 to 95° C. downstream of the upstream pressure control unit in the (feed) conduit. Since the main reactor, in the course of operation, is operated at a constantly rising temperature in order to compensate for the falling catalyst activity, a constantly rising amount of energy can be released to the (feed) stream of matter upstream of the first separation column in parallel. As a result, there is likewise a steady drop in the energy demand in the bottoms circuit, or the heat exchanger of the first separation column incorporated therein.

In one embodiment of the plant, it may be advantageous that the reactor unit comprises a first main reactor and at least one downstream permanent, series-connected postreactor. The particular advantage of the postreactor and its inlet-side temperature control of the stream of matter is that this enables optimized control of the selectivity of the proportions of isomers, because of the preferably higher inlet temperature that differs from the first reactor.

Advantageously, the postreactor is also a fixed bed reactor, or reactor with an immobile catalyst, for example a catalyst bed or catalyst-coated internals.

Advantageously, the immobile catalyst comprises ruthenium, has been doped with ruthenium or is formed therefrom. In an advantageous process variant, especially for achieving a low proportion of trans/trans isomers in the isomer mixture, the main reactor is at a temperature of 90 to 140° C., ideally 95 to 135° C.

It has been found to be particularly advantageous when the ratio of the catalyst masses of the main reactor to the postreactor is in the range from 1.2 to 2, preferably 1.3 to 1.4 and ideally 1.35. It has been found that, surprisingly, it is sufficient to regulate the highly exothermic reaction at the start of the reaction in the main reactor by means of an intensive cooling circuit, and only to adjust the feed temperature in the inlet to the postreactor such that the moderate temperature rise of 30 to 35° C. in the postreactor from the inlet to the outlet has only a limited and readily reproducible influence on the trans/trans isomer content in the stream of matter or in the product, as already set out.

It may be especially advantageous when the main reactor is a fixed bed reactor comprising
a first flow pathway for the reactant mixture or mixture of matter and
a further (closed) flow pathway, i.e. a media circuit for a heat exchange medium, where the second flow pathway incorporates two heat exchangers for indirect heat exchange:
a (main) heat exchanger operating as a cooler and
a (secondary) heat exchanger operating as a heater.

It has been found to be very effective and advantageous to operate the same further flow pathway, or media circulation, for preparation and startup of the main reactor by means of the (secondary) heat exchanger and to operate the cooling of the main reaction in producing operation by the main reactor by means of the (main) heat exchanger.

In a further embodiment of the plant, it may be advantageous that the reactor unit comprises a further main reactor in the form of a fixed bed reactor comprising
a first flow pathway for the mixture of matter and
a further (closed) flow pathway, i.e. a (cooling) media circulation system for a heat exchange medium, and where a valve unit is provided upstream of the two main reactors in the (feed) conduit, by means of which the volume flow rate of the reactant mixture is divisible, conductable and/or completely switchable between the first main reactor and the further main reactor, and wherein the two main reactors are connected
each to one heat exchanger or
collectively to one heat exchanger for the further (closed) flow pathway.

In this case, the two main reactors connected in series are identical or essentially identical in design. In particular, the two main reactors have such dimensions and/or corresponding internals such that the same or substantially the same mass and/or volume of catalyst is present or accommodatable.

The advantage of these two main reactors is that further thermal decoupling of the first reaction phase with very strong exothermicity, the second reaction phase with medium exothermicity and the postreaction with very low exothermicity is possible. Further advantages are that, in the case of the same plant performance, the individual main reactor has smaller dimensions and can therefore be thermally controlled more easily and more homogeneously. At the same time, plant performance is increased because, in the case of maintenance, for example a catalyst changeover, the plant does not have to be shut down completely. For this purpose, the two main reactors are interconnected in such a way that the mixture of matter can also flow through each alone, bypassing the respectively other main reactor (bypass 1).

The hydrogenation in the (isothermal) main reactor with significant cooling allows significant limitation of temperature-induced isomerization. In the (adiabatic) postreactor, a sufficient temperature level is then established in a controlled manner in the incoming stream of matter, especially via heat exchange, and hence isomerization is permitted to specifically afford an on-spec trans/trans content in the product. In a purely isothermal mode of operation of a single main reactor without a postreactor, what would at first be obtained would be excessively low trans/trans contents and a high proportion of unconverted MDA. By virtue of the option of allowing controlled adiabatic hydrogenation of the stream of matter, it is possible to advantageously control the evolution of temperature and hence isomerization in the postreactor.

In a further-improved variant, the interconnection is such that the postreactor can be bypassed in the case of operation of at least one main reactor (bypass 2), but can especially be bypassed in the case of operation of the two main reactors connected in series. In the bypass 2 interconnection variant, the main reactor through which the flow passes second in flow direction at least temporarily assumes the function of the postreactor, such that the plant can be operated without or largely without a drop in performance and/or changes in product quality, especially in the respective proportion of isomers in the isomer mixture.

In a further embodiment of the plant, it may be advantageous that at least one common heat exchanger is disposed in the conduit between the at least one main reactor and the second main reactor. This common heat exchanger is disposed in a central branch of both coolant circuits. The guiding and interconnection of the conduits here is such that, downstream of the common heat exchanger, the cooling medium is first introduced into the first of the two main reactors in which the more strongly exothermic reaction proceeds. The already heated cooling medium is then fed via a conduit into the downstream second main reactor, such that the latter is charged with a feed temperature different from the first main reactor.

The advantage is that it was observed that, surprisingly, in particular, very exact control of the first, highly exothermic reaction phase is crucial for product quality, and so it is possible to dispense with the construction work and control complexity involved in a further, completely independent second cooling circuit. Another reason for this in particular is because complete reaction can be ensured and controlled via control of the feed temperature of the postreactor connected in series downstream of the two main reactors, especially the desired low trans/trans isomer content.

In a plant variant with improved controllability, it may be the case that a heat exchanger (postcooler) which is switchable and controllable as required is disposed in the respective (cross-)conduit of the cooling circuits by which the coolant outlet of the first reactor is connected to the coolant inlet of the second reactor.

The bottoms circulation system of the separation tank is operated at a temperature of 130° C. to 150° C., ideally 135 to 145° C. The great advantage of the separation tank, which is very simple in terms of construction and control, is that the boiling temperature of the mixture is likewise lowered by the lowering of pressure, such that heating in the separation tank is only necessary up to that lowered boiling temperature. Furthermore, by virtue of this measure, about 90% of the solvent present in the reactant mixture, in particular THF, is already removable. There is no need for this purpose for a tops circuit or return stream, as in a column. The stream of matter passed onward to the first separation column thus advantageously has only a remaining solvent concentration of about 20% to 40% by weight, ideally 25% to 35% by weight. It is thus possible for the first separation column to be of smaller design, and operable in a more energy-saving manner owing to the lower mass of the stream of matter.

In this way, the temperature of the stream of matter is additionally lowerable more quickly, and so any rise in the proportion of trans/trans isomers of the PACM in the stream of matter is prevented or reduced.

The solvent-conducting (return) conduit is connected to the conditioning unit and/or to at least one suitable collection tank.

The invention further relates to a process for continuous, catalytic hydrogenation of methylenedianiline (MDA; reactant1) with a hydrogen donor (reactant2), in particular a gaseous hydrogen donor, preferably hydrogen (H2),
wherein the production is effected by means of an industrial plant, wherein the plant is designed according to at least one of the working examples and variants described herein, and wherein the main reactor is operated at a temperature in the range from 80° C. to 150° C., and wherein vapour compression in the intermediate circuit by means of the at least one fan and/or the fan group results in at least an increase in temperature of the medium in the incoming conduit branch by 20° C. to 60° C., ideally from 30° C. to 50° C.

In a further embodiment of the process, a further advantage may be that the at least one main reactor is operated at a pressure in the range from 60 bar to 120 bar, ideally in the range from 70 to 110 bar. In a further advantageous process regime, it may be the case that the pressure in the main reactor is 70 to 100 bar, ideally 80 to 90 bar. Particularly preference is given to a pressure of about 85 to 90 bar.

In a preferred embodiment of the process, it may be the case that continuous catalytic production of methylenebis(cyclohexylamine) is effected, especially production of 4,4′-diaminodicyclohexylmethane (PACM), preferably PACM with low proportions of trans/trans isomers. In a preferred process variant, continuous catalytic production of methylenebis(cyclohexylamine) is effected, especially production of PACM, of the formula (I)

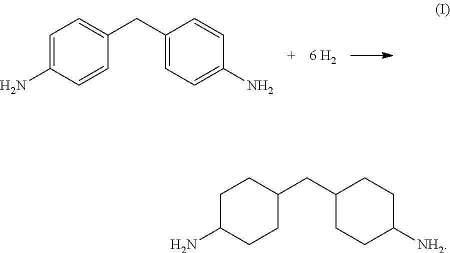 [SMILES: Nc1ccc(Cc2ccc(N)cc2)cc1 ; NC1CCC(CC2CCC(N)CC2)CC1]

In a further embodiment of the process, a further advantage may be that, in addition to the at least one main reactor, at least one catalyst-containing postreactor is also included, where the at least one main reactor and the at least one postreactor are operated at the same or essentially at the same pressure. What is meant here by “essentially” is that the pressure in the main reactor and secondary reactor is at the same level with a difference of up to +/−5 bar. In particular, it has been found to be advantageous when the pressure in the postreactor is slightly lower than in the main reactor, especially about 2 bar to 5 bar lower. The catalyst of the postreactor is likewise an immobile catalyst introduced, for example, in the form of a bed in the postreactor. Advantageously, the catalyst in the postreactor is physically identical or largely identical to the main reactor, where the shape and/or manner of introduction of the catalyst in the postreactor may be different from the main reactor.

In a further embodiment of the process, a further advantage may be that, from time to, the start of the process after renewal or regeneration of the catalyst, to time t₄, the end of the process determined by renewal or regeneration of the catalyst, the operating temperature of the main reactor is increased and the temperature in the stream of matter in the inlet (feed) to the postreactor is maintained or lowered, where the increasing or lowering of the temperature is linear and/or stepwise.

With the plant variant mentioned, it is thus possible for the process to continue until attainment of a low trans/trans content in the PACM of 17% to 25% by weight over time.

In a further embodiment of the process, a further advantage may be that, in the distributor circuit, a multistage increase in pressure is effected in the incoming conduit branch, giving an inlet pressure of 1.5 bar to 5 bar and a temperature of 100° C. to 150° C. downstream of the distributor tank and upstream of the first compressor. Ideally, the (inlet) pressure is 2 bar to 4 bar, with an ideal temperature of 120 to 145° C. Additionally or alternatively, it is advantageous when, downstream of the last compressor and upstream of the first heat exchanger acting as a heat sink, a (final) pressure in the incoming conduit is 10 to 30 bar, ideally 15 to 25 bar, and a temperature is 180° C. to 300° C., ideally 220 to 280° C.

In a further embodiment of the process, a further advantage may be that, in the distributor circuit, a multistage increase in pressure is effected in the first conduit branch, giving a pressure of 3 bar to 30 bar and a temperature of 130° C. to 300° C. downstream of the last compressor and upstream of the first heat exchanger acting as a heat sink.

In a further embodiment of the process, a further advantage may be that the energy introduced into the first evaporator by the collection circuit by means of the intermediate circuit and the at least one incorporated fan and/or the group of x fans is raised
by at least a factor of 1.1 to 2.5 and/or
the output temperature of the tank is raised by 10° C. to 80° C., ideally by 20° C. to 50° C.

“Output temperature” refers here to the temperature in the feed of the distributor tank, in the tops outlet of the tank and/or bottoms outlet of the tank. In a first exemplary embodiment, the distributor tank itself is not separately heated, apart from the heating via the medium coming from the intermediate circuit. In a first embodiment, the tank itself does not have a heating unit or an internal heat exchanger, nor is it connected, for example, to a bottoms circuit. An improvement may be to equip the tank with a separate integrated heater or to connect it to a separate heating circuit, especially in order to supply supplementarily energy as required.

In a further embodiment of the process, a further advantage may be that the MDA (reactant1) comprises a mixture of the at least two isomers: 4,4′ MDA, 2,4′ MDA and 2,2′ MDA, where the proportion of 4,4′ MDA is advantageously in the range from 75 to 98 mol %, preferably 85 to 95 mol %, ideally about 90 mol %. The proportion of 2,4′ MDA in the reactant mixture is advantageously 7 to 15 mol %, preferably 8 to 12 mol %, ideally 9 to 10 mol %. In this case, an advantageous embodiment may be that the solvent is present in a proportion by weight of 40% to 50% by weight based on MDA in the reactant mixture.

Ideally, the proportion of trans/trans PACM is in the range from 15% to 30% by weight, ideally 16% to 25% by weight.

Overall, it is advantageous when the reaction step in the main reactor is implemented isothermally or largely isothermally and the reaction step in the postreactor is implemented adiabatically or largely adiabatically. At time to, the start of process, the catalyst in the postreactor may also be in renewed or regenerated form as well as the main reactor. Advantageously, the cycle for regeneration or renewal of the postreactor is determined autonomously and independently of the main reactor, especially when the abovementioned interaction of the reaction components in the two reactors no longer enables the production of the desired low trans/trans content in the PACM.

In an advantageous embodiment of the process, it may be the case that the temperature of the reactant stream at the inlet of the main reactor is 90 to 140° C., ideally 95 to 135° C.

In a further advantageous embodiment of the process, it may be the case that the pressure in the main reactor is 70 to 100 bar, ideally 80 to 90 bar.

In a further advantageous embodiment of the process, it may be the case that
the temperature at the inlet of the main reactor corresponds essentially to the temperature at the inlet of the postreactor, essentially meaning a range or difference of +/−10° C., and/or the pressure at the inlet of the main reactor corresponds essentially to the pressure at the inlet of the postreactor, essentially meaning a range or difference of +/−5 bar.

Overall, all aspects, advantages and executions relating to plants or mentioned in association with the description thereof are also intended to be applicable identically or analogously to the process, and vice versa, unless stated otherwise and/or there is a technical impossibility associated with analogous application.

The solution according to the invention is described in detail hereinafter with reference to working examples

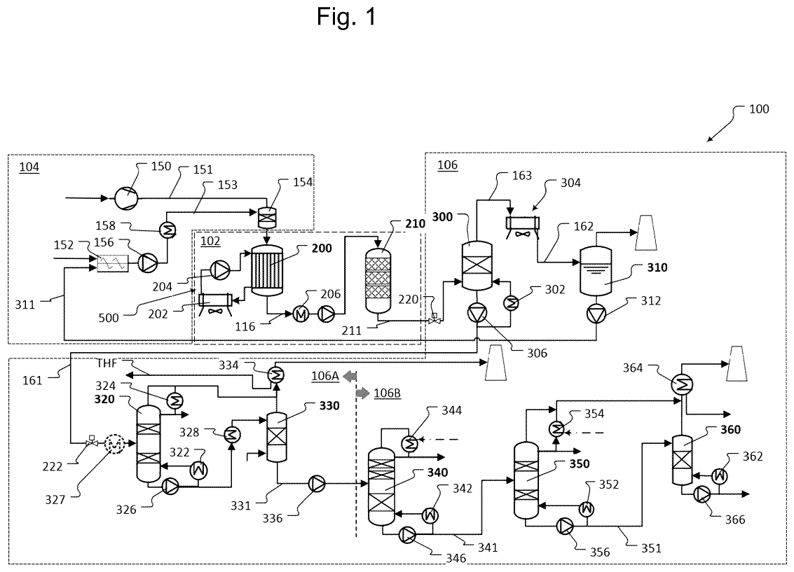

FIG. 1 shows the plant 100 for continuous production of 4,4′-diaminodicyclohexylmethane (PACM) by catalytic hydrogenation of methylenedianiline (MDA; reactant1), especially 4,4′-diaminodiphenylmethane, with a hydrogen donor (reactant2), where the hydrogen donor is supplied in the form of gaseous hydrogen (H2). The plant 100 comprises a conditioning unit 104 for the reactants, a reactor unit 102 and a separation unit 106.

The conditioning unit 104 is framed by dashed lines and comprises (feed) conduits for reactant1 and the hydrogen (reactant2), and also the solvent. In addition, the conditioning unit comprises a compressor unit 150, referred to hereinafter as compressor in the conduit 151 supplying the hydrogen, a mixer 152 in the conduit supplying the solvent and reactant1. Additionally disposed in the conduit 153 supplying the mixture of reactant1 and solvent are a pump 156 and a heat exchanger 158. The conduits 151, 152 open into a mixing vessel 154 disposed upstream of the main reactor 200. The mixing vessel 154 serves for intensive mixing of the reactants, and its outlet forms the inlet for the main reactor 200.

The reactor unit 102 is framed by dashed lines and comprises essentially the main reactor 200, a cooling circuit 500, a postreactor 210 and a heat exchanger 206 in the feed conduit to the postreactor 210. The cooling circuit 500 incorporates a pump 204 and a heat exchanger 202, where the circulating coolant in the main reactor 200 flows around the catalyst material-filled carrier elements. In the example shown, the flow direction toward the catalyst material-filled carrier elements is in cocurrent direction. The postreactor 210 is connected via the conduit 211 to the separation unit 106, i.e. the first separation stage thereof.

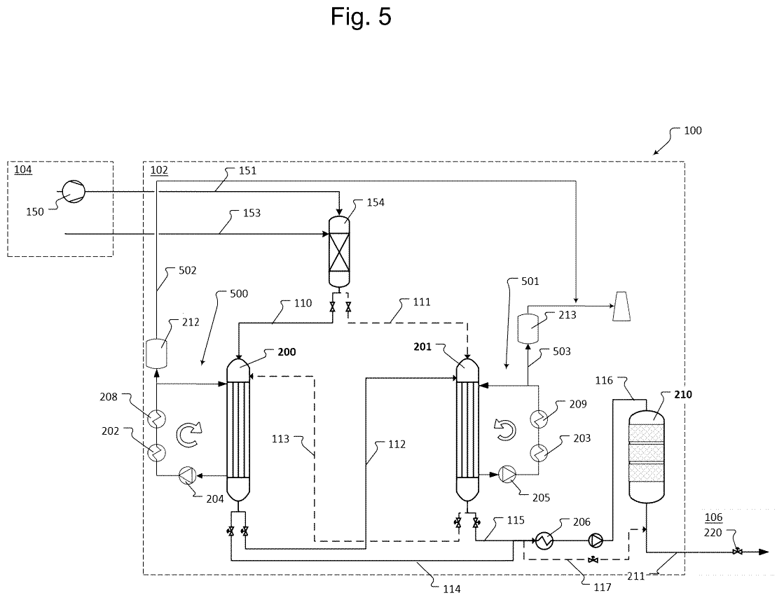

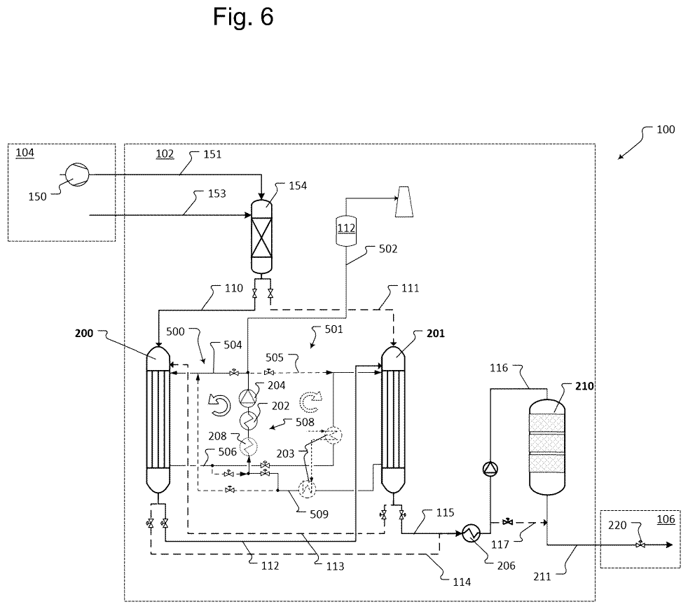

The heat exchanger 202 in the cooling circuit 500 is shown as an air-cooled heat exchanger 202, but may also be alternatively designed in order to control the temperature of the cooling medium in the cooling circuit 500 in indirect heat exchange, for example by means of a flowing cooling medium, such as an oil, water or a brine. The cooling circuit 500, in a plant variant which is not shown, also incorporates a heat exchanger, analogously to the heat exchangers 208, 209 in FIGS. 5 and 6. The latter is operated with a heating medium and serves, in the step of starting up the main reactor 200, to adjust the temperature of the main reactor 200 to about 80 to 100° C., ideally to a temperature of 85° C. to 95° C. In the plant and process example shown, in which one aim is to achieve a minimum trans/trans isomer ratio of about 17% to 23% by weight, the main reactor 200 filled with fresh or regenerated catalyst is preheated to a temperature of about 90° C. by means of the heat exchanger 208. The main reactor 200 is operated at a pressure of 87 to 88 bar.

The separation unit 106 is framed by dashed lines and comprises a plurality of separation apparatuses for separating the solvent, especially in a first separation stage, and the PACM product, especially in a second separation stage, from the rest of the reactant and by-products. The first separation stage (not displayed) comprises a separation tank 300 to which a bottoms circulation system is connected, incorporating a heat exchanger 302 and a pump 306. The top outlet of the separation tank 300 is connected to a heat exchanger 304, a condenser, downstream of which is provided a collecting vessel 310 for the solvent. By means of the heat exchanger 304 (condenser), about 80% of the solvent, THF in this case, is condensed out and could be collected or returned. In the flash stage, an energy requirement of about 1400 kW is required for the heat exchanger 302 in the bottoms circulation system of the separation tank 300.

The condenser 304 is shown as a heat exchanger in the design of an air-cooled apparatus, but may also alternatively be designed to at least partly condense and to cool the vaporous stream of matter at the outset in indirect heat exchange, for example by means of a flowing cooling medium such as cooling water or a medium suitable for thermal integration.

The amounts of energy specified herein are calculated for a plant output of the PACM product in the synthesis reaction mentioned of about 3.37 t/h, and a by-product output of about 0.4 t/h of HB and about 0.05 t/h of LB. The ratio of the mass flow rates of THF to MDA was 4.2 to 4.5.

In addition, the first separation stage of the separation unit 106 for further removal of the solvent comprises a first separation column 320 and a second separation column 330, where the second separation column 330 takes the form of a stripping column. For this purpose, nitrogen (N2) is advantageously used as a stripping medium, which is passed through the column in countercurrent to the stream of matter. By means of the pump 306 disposed in the bottoms outlet of the separation tank 300, the solvent-depleted stream of matter can be diverted via the conduit 161 to the first separation column 320. A pressure control unit 222 is provided in the conduit 161 and is designed as a controllable valve in the example shown. The stream of matter is introduced into the first separation column 320 via a central inlet as shown. Additionally disposed upstream of the first separation column 320 is a (forward) heat exchanger 327 (shown by dashed lines), which constitutes an option for heating of the first separation column 320.

In the first separation column 320, the solvent concentration is reduced from 30% by weight in the feed via the product-rich stream of matter from the conduit 161 to about 2% by weight of residual solvent.

This first separation column 320, equipped with (structured) packings, is connected to a bottoms circulation system incorporating a heat exchanger 322 and a pump 326. In addition, a tops circulation system is disposed at the column top of the first separation column 320, incorporating a heat exchanger 324 designed as a condenser. Two outlets for the solvent-rich stream of matter lead out of the tops circulation system, with one of the two outlets leading into the tops outlet of the downstream separation column 330 (stripping column).

In particular, the solvent separated off in the first separation stage is conductable via the conduit 311 into a collection tank (not shown) and/or the mixer 152 of the conditioning unit 104. In the conduit 211 leading from the postreactor 210 to the separation tank 300, a pressure control unit 220 is provided, designed in the present example as a controllable valve.

The product-rich stream of matter having a residual content of about 2% by weight of solvent (THF) is directed from the bottoms outlet of the first separation column 320 via the conduit 321 into the top of the second column 330, the stripping column. This conduit 321 incorporates a heat exchanger 328. The second column 330 has at least one internal packing, where a gas feed for an inert gas (stripping gas) in particular is disposed below the packing, such that the introduced product-rich stream of matter flows through the second column 330 in countercurrent principle to the stripping gas and is further depleted of solvent. In the plant example shown, nitrogen (N2) is used as stripping gas. The solvent-rich vapour is directed via the tops outlet into a condenser 334 and fed to the condenser 334 together with the vapour stream coming from the tops outlet of the first separation column 320 and introduced into the tops discharge of the second separation column 330. The solvent stream condensed out in the heat exchanger 334 is returned to the conditioning unit 104, wherein the uncondensable portion is led off from the heat exchanger 334 and, for example, fully thermally oxidized.

The product-rich stream of matter is fed from the bottoms outlet of the second separation column 330 to the third separation column 340 via the conduit 331, incorporating the pump 336. The second separation stage 106B of the separation unit 106 serves in particular to separate the LB and/or HB by-products from the PACM product.

The second separation stage comprises essentially three separation columns, where the third separation column 340, the first of the second separation stage, is supplied centrally with the stream of matter. The third separation column is connected to a bottoms circulation system incorporating a heat exchanger 342 and a pump 346. The product-rich stream of matter is directed via the conduit 341 from the bottoms outflow to the fourth separation column 350. In addition, the third separation column 340 is connected to a tops circulation system incorporating a heat exchanger 344. Condensed LB is led off as the first by-product from this tops circulation system.

The product-rich stream of matter is fed centrally to the fourth separation column 350 via the conduit 341. The fourth separation column 350 is connected to a bottoms circulation system incorporating a heat exchanger 352 and a pump 356. In addition, the fourth separation column 350 is connected to a tops circulation system incorporating a heat exchanger 354 in the form of a condenser. The product-rich stream of matter is discharged from the tops circulation system as condensate via the tops outlet. In the present case, according to the example shown, the uncondensed vapour stream and/or gas stream is introduced into the tops discharge of the downstream fifth separation column 360. Subsequently, product is condensed further and discharged via a further heat exchanger 364 (condenser). The stream of matter with a low level of product is discharged via the bottoms outlet of the fourth separation column 350 via the conduit 351 and introduced into the top portion of the fifth separation column 360. This stream of matter is highly enriched with HB, a second by-product. The fifth separation column 360 is connected to a bottoms circulation system incorporating a heat exchanger 362 and a pump 366. Furthermore, the separation column 360 has a tops outlet which leads to the aforementioned heat exchanger 364 in the form of a condenser. In this condenser 364, a further product-rich stream of matter is condensed out as condensate and discharged, with gaseous discharge of the uncondensable portion. In particular, the latter may subsequently be fully oxidized.

With regard to the stream of matter, the reactor unit 102 is operated at a first, high pressure level of about 60 to 120 bar, the first separation stage 106A of the separation unit 106 is operated at a second, low pressure level of 4 to 12 bar, and the first separation column 320 and the second separation column 330 are operated at a third, slightly elevated pressure level of 1.05 to 2.5 bar. In the example shown, the first pressure level is 80 to 90 bar, the second pressure level is 4.5 to 7.5 bar and the third pressure level is 1.1 to 1.2 bar.

With direct temperature control of the main reactor 200 via the cooling circuit 500 in series with the uncooled postreactor 210, surprisingly, a reduced energy requirement compared to the related art and a simplified, more stable process regime has been demonstrated. Without being tied to any specific interpretation, this success is apparent in that the cooling circuit 500 only has to be specifically designed for the very vigorous initial reaction for dissipation of the exothermic heat of reaction, while the still significant postreaction has to be adjusted via the feed temperature by means of the upstream heat exchanger 206 alone. Because the reaction in the postreactor 210 is already highly attenuated, the stream of matter is only heated by about 5 to 15° C. and can be discharged into the separation tank 300 at this level without any problems.

All in all, the figures show internals such as packings, separation planes, support elements etc. in the apparatuses such as reactors, separation columns, vessels etc. by means of the corresponding symbols. These each indicate advantageous embodiments, with regard to the number, type and/or relative position to the respective feed conduit or discharge conduit. For example, the illustration of first separation column 320 thus indicates that, advantageously, the (feed) stream of matter is introduced via the conduit 161 in such a way that at least one (theoretical) separation plane is present in each case between the tops circulation system and the bottoms circulation system. The determination of the specific type and/or number of separation planes is known to the skilled person and can be varied or provided for in an appropriate manner.

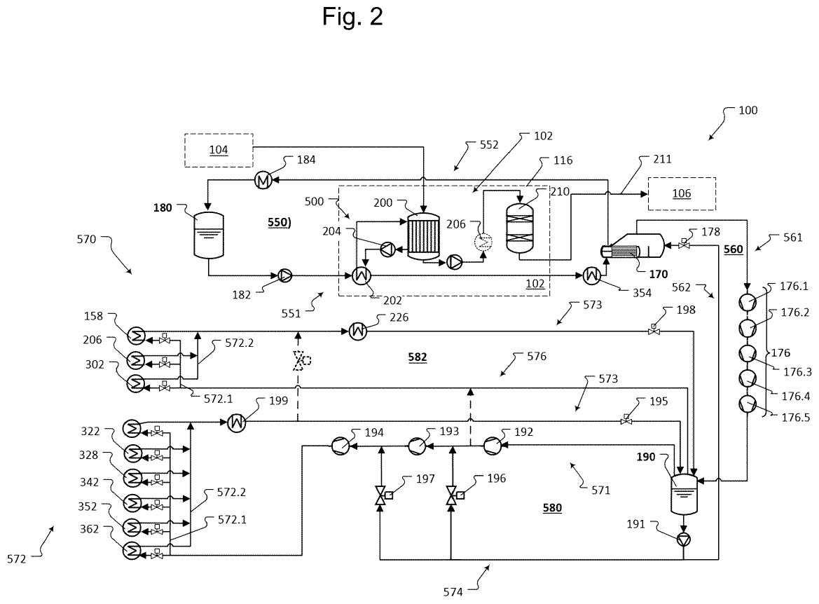

FIG. 2 shows a first embodiment of an energy circuit, wherein three circuits are provided, namely a collection circuit 550, an intermediate circuit 560 and a distributor circuit 570. The collection circuit 550 comprises a collection tank 180, a first conduit 551, an evaporator 170 and a second conduit 552, where the first conduit 551 connects the collecting tank 180 to an inlet of a heat exchanger portion (WT portion) of the evaporator 170 and the second conduit 552 connects an outlet of the WT portion of the evaporator 170 to the collecting tank 180. It is thus only the WT portion of the evaporator 170 that is part of the collection circuit 550.

The first conduit 551 incorporates the heat exchanger 202 of the coolant circulation 500 of the main reactor 200 and the heat exchanger 354 from the tops circulation system of the fourth separation column 350 as heat sources, and a pump 182. In the second conduit 552, in the example shown, the feed to the collection tank 180 incorporates a heat exchanger 182 which is used essentially to set the required feed temperature (cooling) of the collection tank 180 and/or the temperature in the first conduit 551 as required and for control reasons, in order to ensure the temperature gradient for the required cooling of the incorporated heat exchangers 202, 354. The first conduit 551 can also be regarded as a collection or feed conduit and the second conduit 552 as a return conduit. The flowing medium provided in the circuit in the example shown is water, although it would also be possible to use a brine or an oil.

In an advantageous manner, the collection circuit “collects” multiple heat sources in order to transfer the amount of energy thus collected from the medium circulated, here water in particular, to the first evaporator. This significantly reduces design complexity compared to a direct interconnection of the heat exchangers in question.

The intermediate circuit 560 comprises the aforementioned evaporator 170, a first (incoming) conduit 561, a fan group 176, a returning conduit 562 and a pressure control unit 178. The tank portion (K portion) of the evaporator 170 is part of the intermediate circuit 560, which also incorporates the distributor tank 190 in open form. This means that the heat exchange medium in the intermediate circuit 560 also flows through the distributor tank 190 and the distributor circuit 570. The heat exchange medium used is advantageously water or water vapour. The pressure control unit 178 is disposed upstream of the inlet of the return conduit 562 into the K portion of the evaporator 170. In the example shown, the fan group 176 comprises five fans 176.1 . . . 176.5 that are incorporated into the feed conduit 561 and connected in series to one another.

The first, incoming conduit conducts the water vapour, which is superheated in the five compression stages from a starting temperature downstream of the evaporator 170 of about 90° C. to about 135° C. The pressure is raised by the fan group 176 from about 0.5 to 1 bar up to about 3 bar. The media stream, in particular the vapour portion, is accommodated in the distributor tank 190, circulated in the distributor circuit 570 and cooled therein by release of heat to the heat exchangers working as a heat sink, such that the water vapour is at least partly condensed out and flows back to the distributor tank 190. By means of the (bottoms) pump 191 and the return conduit 562 and the pressure control unit 178, the medium is returned to the WT portion of the evaporator 170 and evaporated again.

In addition to the possibility of significantly raising the temperature level, another advantage of the intermediate circuit is that water or water vapour is utilizable as heat exchange medium in the intermediate circuit and in the distributor circuit. The use of water/steam in the coupled two circuits has the advantage that water/steam is already present in the plant 100 as a heat exchange medium, and so no additional risks arise with regard to product contamination due to leaks.

In principle, it would also be possible to use another suitable heat exchange medium, such as especially an alcohol, especially an alcohol having 1 to 6 carbon atoms (C1-6 alcohol). This may be, for example, methanol, ethanol, isopropanol, propanol, (tert-, iso-)butanol.

The distributor tank 190 incorporated into the intermediate circuit 560 is connected to the distributor circuit 570 which comprises an HP distributor circuit 580 and an LP distributor circuit 582. The HP distributor circuit 580 comprises a first, incoming conduit branch 571, a second distributor conduit branch or conduit section 572 and a third return conduit branch 573, also called (return) conduit. A tops discharge of the distributor tank 190 leads into the first conduit branch 571, where, in the exemplary embodiment shown, three series-connected compressors 192, 193, 194 are incorporated into the first conduit branch 571. Furthermore, the HP distributor circuit 580 comprises a (bottoms) conduit 574 which opens from a bottoms outlet of the distributor tank 190 via two conduit nodes (intermediate feed) into the first conduit branch 571, where a pump 191 is incorporated in the (bottoms) conduit 574. The first introduction of heating medium is effected via the first conduit node between the first compressor 192 and the second compressor 193; the second introduction of heating medium is effected between the second compressor 193 and the third compressor 194. A controllable valve 196, 197 is disposed in each case upstream of the respective, unidentified conduit nodes.

Also connected to the distributor tank 190 is the distributor circuit 570 with an LP distributor circuit 582 parallel to the HP distributor circuit 580. The LP distributor circuit 582 likewise comprises a first, incoming conduit branch 576, a second distributor conduit branch or conduit section 572 and a third return conduit branch 573, also called (return) conduit. The incoming conduit branch 576, in the example shown, does not incorporate a compressor, such that the water vapour from the distributor tank 190 is introduced at about 3 bar and at a temperature of about 133° C. into the heat exchangers 158, 206, 302 that act as a heat sink. By means of a pressure control unit 198 disposed in the return conduit 573, expansion to the pressure levels that exist in the distributor tank 190 or the required pressure gradient in the LP distributor circuit 582 is controlled.

The distributor conduit section 572 incorporates heat exchangers to be supplied as heat sinks; these are the heat exchangers 322, 328, 342, 352 and 362 which are connected in parallel to one another and are all heat exchangers from bottom circuits of the separation columns. The parallel distributor conduit section 572 of the LP distributor circuit 582 incorporates further heat exchangers to be supplied as heat sinks; these are the heat exchangers 158, 206, 302 connected in parallel to one another.

A central distributor conduit 572.1 leads to the respective heat exchangers of a group and a central collection conduit 572.2 is fed from the respective heat exchangers of a group and leads into the respective (return) conduit 573 of the HP or LP distributor circuit. The distributor conduit section 572 is connected to the distributor tank 190 via the (return) conduit 573. Each of these (return) conduits 573 incorporates a heat exchanger 199, 226 and a pressure control unit 195, 198, by means of which the final pressure of the third compressor 194 of 20 bar or the vapour pressure of the LP distributor circuit is reduced again to the level of the distributor tank of about 2.5 to 3.5 bar. The (return) conduits 573 are each connected to an inlet of the distributor tank 190.

The evaporator 170 shown is what is called a kettle-type evaporator, which comprises a heat exchanger portion (WT portion) which is closed to a first flowing medium and a tank portion (K portion) which is open to another flowing medium. The WT portion has an inlet and an outlet, where the heat exchange medium is guided in closed channels or pipes, for example at least one shell-and-tube system. The K portion has at least one inlet and one (tops or vapour) outlet each, where the distributor tank 190 may also have a (bottoms) outlet. Liquid medium introduced, here via the return conduit 562, is heated by means of the WT portion or the associated heat exchanger and at least partly evaporated. The K portion may be in two parts or have two subspaces. There is a central K portion here, into which the heat exchanger of the WT portion also protrudes and the energy input into the K portion takes place. The further subspace is disposed in a lateral or outer K portion. This may advantageously, but not necessarily, be formed by internals as a zone calmed with respect to the liquid medium and/or is determined in that the heat exchanger of the WT portion does not protrude into this subspace.

The water-conducting distributor circuit is at a temperature of 133° C. and 3 bar at the incoming conduit branch 571 of the HP distributor circuit 580 immediately downstream of the distributor tank 190. Downstream of the first compressor 192 the pressure is 6 bar, which is achieved with a power consumption of 71 kW, downstream of the second compressor 193 the pressure is 12 bar, which is achieved with an electrical power consumption of a further 80 kW, and downstream of the third compressor 194 the pressure is 20 bar at a temperature of 250° C., achieved by further electrical power consumption of 71 kW for the third compressor 194.

For supply of the heat exchangers of the HP distributor circuit 580 shown in FIG. 2, which are in particular the bottom heat exchangers of the separation columns, it is necessary to provide a very high temperature level, and so the circulating medium has to be cooled again via the demand and control heat exchanger 199. In the example shown, the required cooling capacity is about −20 kW, which has to be discharged in the (return) conduit 573 upstream of the pressure control unit 195 or prior to entry into the distributor tank 190.

FIG. 2 shows two further execution variants shown in the form of dashed lines. According to the first variant, the LP distributor circuit 582 branches off not from the distributor tank 190 but downstream of the first compressor 192, and so is operated at the elevated first pressure level. According to the second variant, which is combinable with the first, the return conduit 573 downstream of a pressure control unit (also shown by dashed lines) is introduced into the return conduit 573 of the LP distributor circuit 582 and in this way directed to the distributor tank 190. In this case, it is advantageously possible to dispense with one of the safety and demand heat exchangers 199, 226 in the return conduit 573.

A significant advantage of the distributor circuit can be considered to be that a central steam circuit has been created and hence steam can be generated centrally and distributed to all heat exchangers that operate as consumer (heat sink), instead of directly interconnecting heat sources and heat sinks. Furthermore, the distributor circuit as a central steam circuit, if required (for example when starting up the plant), can be fed at least temporarily by means of an alternative heat or steam source.

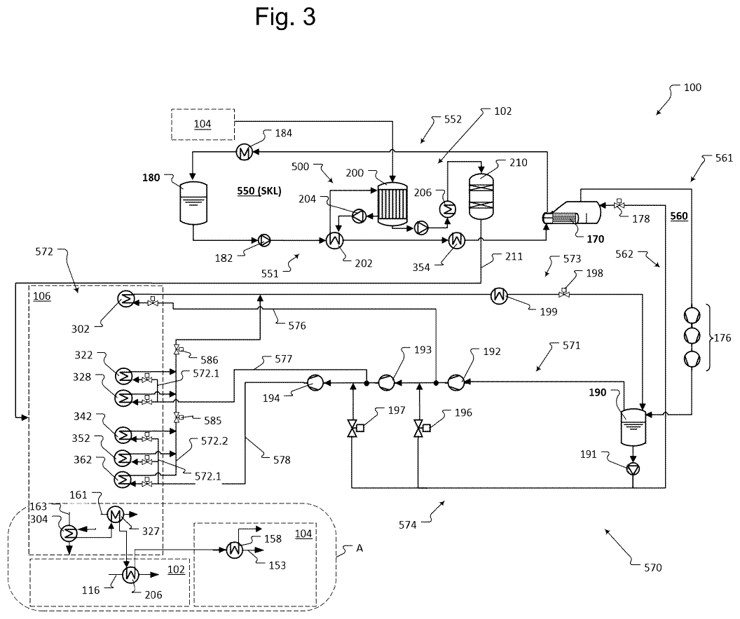

FIG. 3 shows an embodiment in which the collecting circuit 550 and the intermediate circuit 560 are formed analogously to FIG. 2. The distributor circuit 570 differs from the embodiment according to FIG. 2 in that the conduit section 572 via which energy is distributed to the heat exchangers operating as heat sinks is divided into that of the HP distributor circuit 580 and two subsections of the LP distributor circuit 582, these three subsections being connected in parallel to one another:
the HP distributor section, connected downstream of the third compressor 194,
a mid-pressure subsection (MD subsection) of the LP distributor circuit 582, connected downstream of the second compressor 193, and
a low-pressure subsection (LP subsection) of the LP distributor circuit 582, connected downstream of the first compressor 192.

Each subsection has a dedicated distributor conduit 572.1 and is connected to the distributor tank 190 via an incoming conduit or conduit branch 576, 577 and 578 as branch from the incoming conduit branch 571. Furthermore, a common collecting conduit 572.2, into which each subsection feeds, leads into a common return conduit branch 573. The heat exchangers within each of these subsections are connected in parallel to one another, if two or more heat exchangers are included.

In this case, the conduit node with which the first (output) conduit 576 of the LP distributor circuit 582 branches off from the central conduit branch 571 is disposed between the first compressor 192 and the second compressor 193, the conduit node with which the second (output) conduit 577 of the LP distributor circuit 582 branches off from the central conduit branch 571 is disposed between the second compressor 193 and the third compressor 194, and the third (output) conduit 578 which constitutes as last portion of the central conduit branch 571 and forms part of the HP distributor circuit 580 is connected downstream of the third compressor 194. In this case, it is solely the first (output) conduit 576 that supplies the heat exchanger 302, the LP subsection of the conduit section 572, the second (output) conduit 577 that supplies the two parallel-connected heat exchangers 322, 328, the MD subsection of the conduit section 572, and the third (output) conduit 578 (HP distributor circuit 580) that supplies the three parallel-connected heat exchangers 342, 352, 365, the HP subsection of the conduit section 572. Thus, the three (output) conduits 576, 577 and 578 each have different pressure levels and different temperature levels. Liquid medium is fed into the central conduit branch 571 via the (bottoms) conduit 574 or via the branches, analogously to the embodiment of FIG. 2, where the conduit nodes of the three (output) conduits are disposed in flow direction upstream of the conduit node of the respective inlet from the (bottoms) conduit 574.

The lower subsection in the illustration, incorporating the heat exchangers 342, 352, 362 (HP distributor circuit), is kept via the pressure control unit 585 in the collecting conduit 572.2, and the central subsection (LP distributor circuit) incorporating the heat exchangers 322, 328 is kept at an autonomous pressure level via the pressure control unit 586 in the collecting conduit 572.2. Stepwise expansion thus takes place in the collecting conduit 572.2.

The three subsections of the conduit section 572, by means of which the energy is distributed to heat exchangers operating as heat sinks, lead into a common (return) conduit 573 and into the distributor tank 190.

The advantage of this embodiment variant over that from FIG. 2 is the significantly lower energy requirement for the second and third compressors 193, 194.

The water-conducting distributor circuit likewise is at a temperature of about 133° C. and 3 bar at the incoming conduit branch 571 immediately downstream of the distributor tank 190. Downstream of the first compressor 192 the pressure is about 6 bar, which is achieved with a power consumption of likewise 71 kW, downstream of the second compressor 194 the pressure is likewise about 12 bar, which is likewise achieved with an electrical power consumption of 80 kW, and downstream of the third compressor 194 the pressure is analogously 20 bar at a temperature of 250° C., but achieved by electrical power consumption of only 42 kW for the third compressor 194. This advantage arises because the volume flow rate in the second and third compressors 193, 194 is greatly reduced. In addition to the energy benefit, this means that the third compressor 194 can be of structurally significantly smaller design, which furthermore generally likewise improves maintenance and operation and/or installation. Another advantage of the embodiment according to FIG. 3 is that, by virtue of the graduated compression and branching from a part-circuit of the LP distributor circuit 582 in the distributor circuit 570, only one fan group 176 with three fans 176.1, 176.2, 176.3 has to be provided in the intermediate circuit 560 to reach the temperature level for the heat exchangers operating as a heat sink. In the present case, the LP distributor circuit 582 may also be regarded as consisting of two part-circuits.

For supply of the heat exchangers shown in FIG. 3, which are in particular the bottom heat exchangers of the separation columns, it is necessary to provide a very high temperature level, and so the circulating medium has to be cooled again via the demand and control heat exchanger 199.

In principle, the LP subsection incorporating the heat exchanger 302 could also incorporate the heat exchangers 206 of the reaction unit 102 and/or the heat exchanger 158 of the conditioning unit 104.

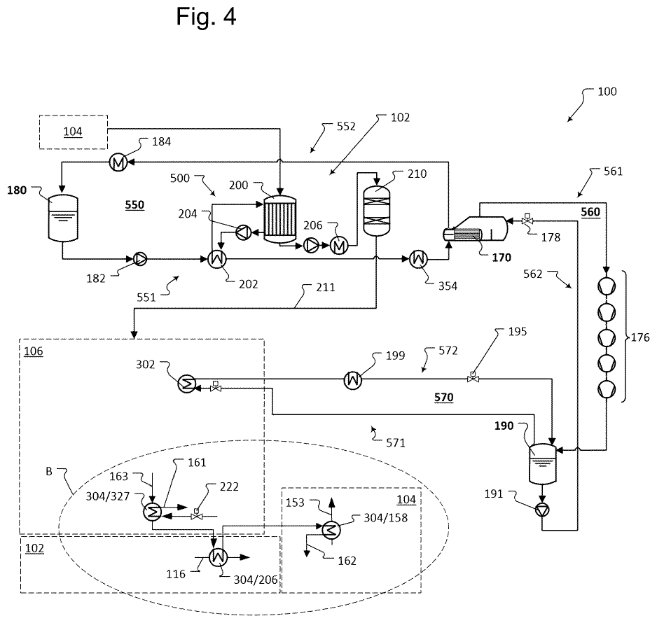

In the example shown in FIG. 3, the heat exchangers 304 (condensers) as heat source are coupled directly, i.e. series-connected, via media conduits to the heat exchangers 158, 206 and 327 operating as a heat sink. In this case, heat sinks incorporated are the heat exchanger 327 in the (feed) conduit 161 to the first separation column 320, the heat exchanger 206 in the feed to the postreactor 210 and the heat exchanger 158 into the (feed) conduit 153 upstream of the main reactor 200. This option of direct thermal coupling via media conduits is shown in the lower box marked A in FIG. 3. This selective direct interconnection to the serial EC by means of media conduits and distribution of the heat from the condenser 304, compared to the incorporation of the heat sinks into the distributor circuit 570 according to FIG. 2 for example, as described above, results in an increase in efficiency of 30% to 50%. The benefit is achieved especially when at least one EC is an integrated material-based EC by coupling heat exchanger 304 to the (forward) heat exchanger 158 and/or the (feed) heat exchanger 327 (not shown; analogous to FIG. 4) upstream of the first separation column 320 of the first separation stage 106B.

The working example as shown in FIG. 4 can be viewed as a structurally simple solution which already achieves 60 to 80% of the energy saving of the plant as achieved with the previous solutions of complete or largely complete power interconnection. In this case, the distributor circuit 570 comprises only one circuit. This distributor circuit lacks integration of a compressor in the incoming conduit branch 571. The pressure level is 3 bar and the temperature is 133° C.

The distributor tank 190 is analogously incorporated into the intermediate circuit 560, the fan group 176 of which has five individual fans 176.1 . . . 176.5.

The motivation for this embodiment may be to minimize the need for electrical conduction for the operation of compressors and nevertheless achieve substantial energy savings and, in particular, avoidance of primary forms of energy, such that only the single heat exchanger or a group of few heat exchangers are coupled and supplied by these routes with a comparatively low temperature level, i.e. at a temperature of up to 130 to 150° C.

Another option shown in the example of FIG. 4 is an improvement or extension. In the first option, the heat exchanger 304 (condenser) is connected to the heat exchangers 158, 206 and 327 as heat source analogously to detail A in FIG. 3. The option of direct interconnection of individual heat exchangers is shown in the frame labelled B in FIG. 4. In contrast to FIG. 3 and frame A, there is direct interconnection of the streams of matter in the conduits 163/161, 163/116 and 163/153 for indirect heat exchange (series integrated material-based EC) in one heat exchanger 304/327, 304/206 and 304/158 in each case. In other words, the required cooling capacity in the tops conduit 163 is provided in the example shown in the three heat exchangers mentioned, and the streams of matter in the said conduits 161, 116, 153 are heated in parallel. In an embodiment which is not shown, it may be the case that an (on-demand) heat exchanger is provided to ensure complete condensation in the (tops) conduit 163, 162.

It has been found that, surprisingly, an integrated material-based EC in particular, through the integration of the heat exchangers 304/327 in one component and the arrangement of the pressure control unit 222 upstream thereof in the conduit 161, leads to substantial cooling of the stream of matter in the tops conduit 163 and also significantly reduces the energy demand of the (bottoms) heat exchanger 322 of the first column 320. By means of the pressure control unit 222, by expansion in the feed 161 to the heat exchanger 304/327, it is possible to lower the temperature level to such an extent that a temperature difference of about 5 to 10° C. exists between the (tops) conduit 163 and the (feed) conduit 161.

This direct interconnection of the streams of matter and distribution of the heat from the condenser 304, compared to the incorporation of the heat sinks into the distributor circuit 570 according to FIG. 2, as described above, results in an increase in efficiency of 30% to 50%.

What is meant in the present context by “an increase in efficiency”, unless stated otherwise, is a lower energy consumption. The reference point will be apparent from the context and may be based on the plant, the plant section respectively described or the improved apparatus, for example via integration of two heat exchangers into a single one.

The second option involves direct coupling of two or more heat exchangers, here the (upstream) heat exchanger 327 of the separation column 320 in the (feed) conduit 161 to the (forward) heat exchanger 206 of the postreactor 210 in the conduit 116 (shown by dashed lines).

The embodiment, in which only the heat exchanger 302 is provided in the distributor circuit 570, constitutes a particularly simple and efficient transmission system to cover a significant portion of the heat requirement with high efficiency. This integration alone can increase efficiency by about 40% compared to direct heating of the heat exchangers. The direct interconnection of the heat exchangers 304 as heat source with the heat exchangers 206, 158 as (consumers) heat sinks leads to an increase in efficiency of about 28%, i.e. energy saving of 28%. Thus, it is possible by both measures, the distributor circuit 570 and the direct interconnection of the condenser 304 with the heat exchangers 206, 158, to achieve an increase in efficiency of about 70%.

One of the two options according to the two details A (FIG. 3), B (FIG. 4) may alternatively be provided in the embodiments according to FIG. 3 or 4.

FIG. 5 shows an improved variant of the reactor unit 102. Two main reactors 200, 201 here are switchably connected to one another in series. Some of the valves/valve units that can be controlled by open-and/or closed-loop control are shown in FIG. 5; others can be provided if required by the person skilled in the art in order to ensure safe operation of the two main reactors 200, 201. The two main reactors 200, 201 are each incorporated into a cooling circuit 500, 501, with which the fixed bed reactor 200, 201 is kept in each case at a permissible, cooled reaction temperature. In the interconnection of the main reactors 200, 201 illustrated and shown with solid lines, the flow passes first through the first main reactor 200 via the conduit 110 coming from the mixing vessel 154 and the first part of the reaction takes place in this main reactor 200.

Downstream via a lower outlet and the conduit 112, the mixture of matter is fed into the top of the second main reactor 201 and leaves it via a bottoms outlet and conduit 115 into the common conduit 116 as a feed into the common postreactor 210. The common conduit 116 incorporates a heat exchanger 206, by means of which the temperature level required for the postreactor 210 can be ensured. The sequence of the flow through the two main reactors can also be reversed, from the main reactor 201 to the main reactor 200. For this purpose, in an analogous manner, the flow to the main reactor 201 first passes through the conduit 111, with the conduit 110 to the top of the main reactor 200 closed. The mixture of matter leaves this fixed bed reactor via a bottoms outlet and the (intermediate) conduit 113 which is connected to the top of the main reactor 200, with the conduit 115 closed. Finally, the mixture of matter leaves the main reactor 200 via a bottoms outlet and conduit 114, which opens analogously into the common conduit 116 and thus leads to the postreactor 210. Each of the two cooling circuits 500, 501 has a conduit 502, 503 which branches off and is guided in each case through a container 212, 213, which serve as pressure equalization vessels, and further to a chimney and/or a complete oxidation unit. The conduits and valves/valve units are present and designed in such a way that each of the main reactors 200, 201 can be operated alone and the stream of matter can completely bypass the respectively other main reactor. The flow direction of the two cooling circuits 500, 501 is symbolized by an arrow, where the two cooling circuits 500, 501 are advantageously identical or substantially identical, since the two main reactors 200, 201 are operated alternately with respect to the flow direction of the reactant stream or the stream of matter as the first or second main reactor.

However, the cooling circuits 500, 501 are designed and controllable in such a way that, depending on the process-related requirements, such as, in particular, product management and/or product quality, autonomous cooling outputs or cooling functions are achieved in each case. This relates in particular to the volume flow per unit time of cooling medium and/or the temperature level or the permissible heating of the respective cooling medium.

FIG. 5 also shows an optional conduit 117 as a dashed line (bypass 2), by which the postreactor 210 can be bypassed if, for example, it has to be maintained and/or the catalyst charge has to be renewed. In this case, the temperature at least of the second main reactor in flow direction of the stream of matter is controlled such that complete reaction is ensured with the desired product quality, in particular the desired proportion of the respective isomers. The conduit branch of the conduit 117 may be provided upstream or downstream of heat exchanger 206 in flow direction, advantageously upstream of heat exchanger 206, in order also to be able to bypass it if necessary, and to be able to undertake necessary maintenance operations while the plant is running.

The main reactor 200 can be bypassed in operation of the main reactor 201 shown on the right in the picture via the conduits 111, 115 and 116. The main reactor 201 can be bypassed in operation of the main reactor 200 shown on the left in the picture via the conduits 110, 114 and 116, such that there is no flow through the (cross-) conduits 112, 113 between the two main reactors 200, 201 in each case.

The cooling circuits 500, 501 also (optionally) incorporate heat exchangers 208, 209 in the plant variant shown. These are operated with a heating medium, especially steam, and serve for (pre)heating the reaction temperature of the respective main reactors 200, 201 in the startup step of the main reactors 200. In the plant and process example shown, as already set out for FIG. 1, with a view to the aim of achieving a minimum trans/trans isomer ratio of about 17% to 23% by weight, it is advantageous when the newly catalyst-filled main reactor 200, 201 is preheated to a temperature of about 90° C. by means of the respective heat exchanger 208, 209, or the respective newly filled main reactor 200, 201 is correspondingly preheated.

The preheating to typically 85 to 95° C. makes it possible for the reaction in the case of the present catalyst to start immediately, without or substantially without recycling streams of the mixture of matter from the process until the desired reaction temperature has been attained.

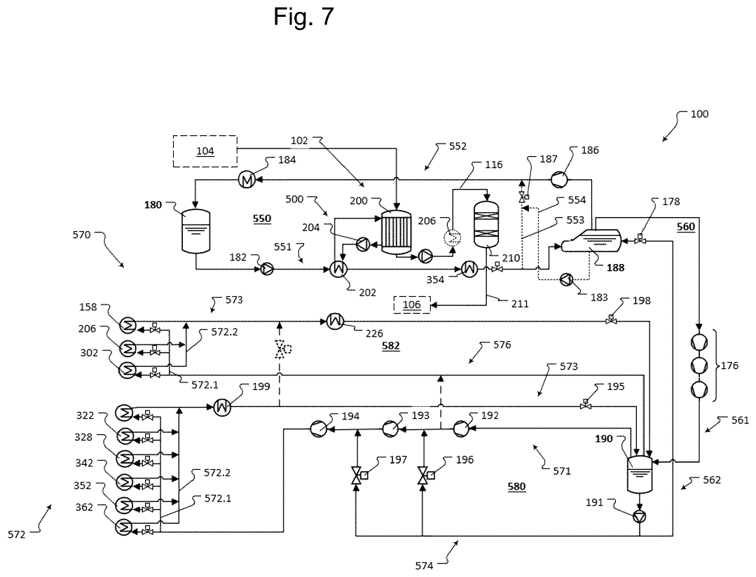

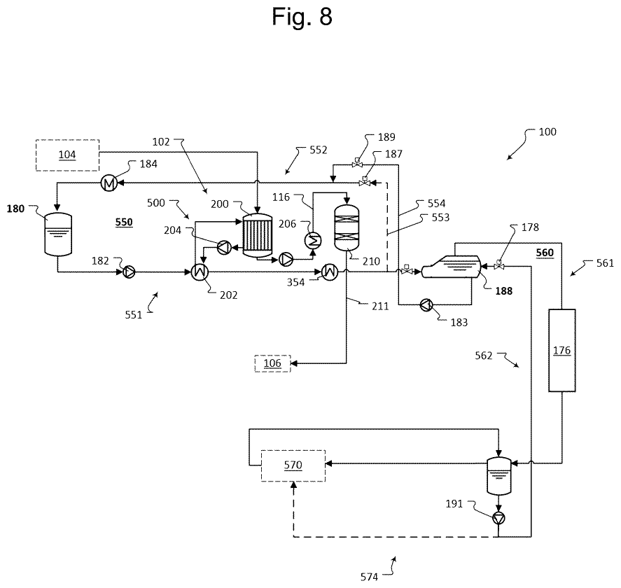

The variant of the switchable main reactors 200, 201 as shown in FIG. 8 differs from that according to FIG. 7 in that the cooling circuit 500 of the first main reactor 200 is likewise connected in series with the cooling circuit of the second main reactor 201. In this case, only one (cooling) heat exchanger 202 and only one pump 204 is provided for the common cooling and the media circuit, and so the flow through the common (central) conduit branch 508 in both cooling circuits 500, 501 is generally constant and only in one direction, irrespective of the interconnection of the two main reactors 200, 201. Of course, the “central” conduit branch need not in fact be disposed between the two main reactors 200, 201. The variant is shown as solid lines, in which the first main reactor 200 (on the left) is first fed with the reactant mixture via the conduit 110 and also the introduction (of cooling media) downstream of the heat exchanger 202 and the pump 204 is first effected via this main reactor 200. The conduits that do not carry media in this circuit or conduits that carry the mixture of matter are shown by dashed lines. In these variants too, it is possible to completely bypass the respectively other main reactor with the mixture of matter and/or the (cooling) medium. For example, in the process of filling one of the two main reactors 200, 201, the respectively other main reactor can thus continue to be operated at maximum output. The bypass of the stream of matter or of the respective main reactor 200, 201 is analogous to FIG. 7.

In the example circuit shown, the flow passes through the central conduit branch 508 and the heat exchanger 202 and is directed via the conduit 504 in cocurrent into the media space of first main reactor 200, blocking conduit 505 that leads to the common conduit node coming from the second main reactor 201. The medium leaves the first main reactor 202 via the conduit 506 at a low-level outlet and is directed to a high-level inlet into the media space of the second reactor 201 (cross-conduit). The medium likewise flows through the media space of the second main reactor 201 in cocurrent and leaves it at a low-level outlet via the conduit 509, from which a branch again flows into the central conduit branch 508, and so flow through the circuit can continue. Similarly, the flow passes firstly via the conduit 505 through the main reactor 201 shown on the right when the conduit 504 is blocked. The medium then leaves the media space of the second main reactor 201 via the conduit 509 and is directed into the media space of the other main reactor 200 at a high-level inlet. The outlet of the media space at a low-level point leads into the conduit 506 and thence via a branch into the central conduit branch 508.

The particular advantage of the switchable main reactor 200, 201 is the possibility of operating the main reactor, through which the flow passes first, at a higher temperature because the catalyst is already partly exhausted and inactivated, essentially without adversely affecting (i.e. increasing) the trans/trans isomer ratio. At the same time, more significantly heated cooling medium is obtained in the respective (circulation) heat exchanger 202, 203 of the main reactor 200, 201 through which the flow passes first. Because of the higher temperature, this hotter heat exchange medium can be better used in the plant for integrated media-based EC and/or for customary heat exchange with a reactant stream of matter.

FIG. 6, analogously to FIG. 5, shows the optional conduit 117 as a dashed line (bypass 2), which allows the postreactor 210 to be bypassed, where the conduit 117 branches off downstream of the (forward) heat exchanger 206 of the postreactor 210.

FIG. 6 also shows a plant variant (dashed line) whereby higher safety or a higher degree of freedom of temperature management is obtained in the respective (cross-)conduit 504, 505 of the cooling circuits 500, 501 in that switchable and controllable heat exchangers 203 (feed coolers) are arranged as required. In the working example shown, the two heat exchangers 202 are connected in series, since, because of the respectively inactive (cross-)conduit, the conduit 505 in the example shown, no heating takes place at the site of installation for the heat exchanger 202. The cooling media conduit or the cooling media circuit of the heat exchangers 202 can be operated in an advantageous variant in a sidestream or via a secondary or auxiliary circuit via the pump 204.

The great advantage of this sidestream or secondary/auxiliary circuit of a coolant via the heat exchangers 203 is that this additional cooling output only needs to be called on as required and, with a low degree of complexity, a greater extent of closed-loop temperature control as required is possible in the respectively second of the two series-connected main reactors.

FIG. 7 also shows a plant variant designed analogously to FIG. 2 in particular. In contrast to the preceding embodiments and variants of FIGS. 2 to 4, the collection circuit 550 is likewise coupled to the intermediate circuit 560 via a distributor tank 188. The heated stream of matter in the incoming conduit 551 is thus discharged openly into the interior of the distributor tank 188, analogously to the distributor tank 190, such that heating and evaporation in the distributor tank 188 are possible via the introduction of the media flow, hot water here, from the conduit 551 via an expansion valve not specified in any further detail. Also provided in the return conduit 552 of the collection circuit 550 is a compressor in order to ensure a defined return of vapour to the collection tank 180. Also shown as dashed lines are conduit 553, 554, as options for improved closed-loop control of the fill level in the distributor tank 188. The (media) conduit 553 is a bypass conduit to the distributor tank 188, and the (bottoms) conduit 554, also incorporating a pump 183, is a withdrawal pathway for closed-loop control of a defined fill level and hence also the feed rate of hot medium from the incoming conduit 551.

In the embodiment of FIG. 8, an alternative to FIG. 7, only a tops discharge or tops conduit is provided in the distributor tank 188 for the connection of the conduit 561, not for the return conduit 552 of the collection circuit 550. The incoming conduit 551 has an analogous connection to the distributor tank 188, which leads via an expansion valve not specified in any further detail. In this embodiment, the (bottoms) conduit is incorporated into the return conduit 552 via a conduit node downstream of the pressure control unit 187; in order to establish the required pressure level, a pressure control unit 189 is additionally provided in the (bottoms) conduit 554. In regular operation, there is no flow through the (bypass) conduit 553. Here, too, there is the option if required of specifically controlling the fill level and also the feed rate in the distributor tank 188. A further advantage of this solution is that the (bottoms) pump 183, working in the fluid medium, is able to work with lower energy consumption against the suction conduit of the fan group 176 than is required for the compressor 186 according to FIG. 7.

The (forward) heat exchanger 206 is described primarily in the present context and is in some cases shown in an EC in which it operates as a heat exchanger 206 primarily as a heat sink, meaning that the stream of matter conducted therein is heated. Because of the dependent mode of operation of the postreactor 210 with adaptation to the main reactor 200, 201, cooling of the stream of matter in the (feed) conduit 116 upstream of the postreactor 210 may be required at least temporarily, in particular permanently, because about 10% to 20% of the conversion occurs in the (adiabatic) postreactor, such that the stream of matter is heated up to about 140° C., measured in the outlet. Thus, independently of the embodiments and variants of the plants and of the process that are described herein, it is possible to provide an adapted EC for cooling (heat exchanger 206 operates as a heat source). Alternatively or additionally, supplementary cooling may be provided via a modified or additional EC.

Overall, a multitude of customary open-loop and closed-loop control elements that are known to the person skilled in the art and are necessary or advisable for an advantageous process regime are not shown, such as sensors (flow, temperature, pressure etc.), displays, setting and control elements (especially valves, further pumps, compressors), collecting vessels etc., and should be added if required. In particular, when “a” pump or “a” compressor is mentioned, this also means customary redundancies from at least two parallel aggregates, especially of two parallel pumps or two parallel compressors. In an analogous manner, “a heat exchanger” should not be understood to be limiting and, with the respective local heat exchange function, also means arrangements of heat exchangers that are connected in series or in parallel, including redundant heat exchangers, which in this context does not mean interconnections with at least one further heat exchanger and a locally different heat exchange function.

All heat exchanges and condensers are fundamentally designed such that indirect heat transfer occurs and there is no physical mixing of reactants, product, by-products and/or solvent with the (heating/cooling) medium, such as gas, steam, water, oil, brine etc., unless stated otherwise.

Even if components such as valves, pressure control unit, isolators etc. are shown individually or separately in the present context for simplified description and to some degree were not mentioned individually, this should not be read in a limiting manner; instead, the person skilled in the art is able to combine two or more of these components in a valve unit or control unit as required or provide multiway valves instead.

In the present case, what is meant by “upstream” or “downstream” is the arrangement and/or flow direction of the product-rich stream of matter, unless stated otherwise. Furthermore, “media”, “media stream”, “media conduit”, etc., always means a heating or cooling medium or the associated conduit, unless stated otherwise.

The terms “bottoms outflow”, “bottoms output”, “bottoms outlet” or “bottoms discharge” are to some degree used synonymously, as are analogously the terms “tops outflow, output, outlet or discharge”.

Furthermore, the expression “heat exchanger operated as a condenser” should be interpreted broadly, and also means incomplete condensation or cooling of the supplied stream of matter, and so a “heat exchanger operated as a condenser” is also synonymously named “condenser” in some cases.

The term “bottoms pump” means a pump incorporated into a bottoms circulation system of an apparatus (separation column, vessel, etc.) and/or a pump downstream of the bottoms outlet of an apparatus, which is intended to convey liquid stream of matter.

For all embodiments and variants of the plant and of the process, it may generally be the case that the second separation column 340 and the third separation column 350 are executed as a single column, in particular as a dividing wall column (not shown). In this case, the separation functions of the second separation column 340 and of the third separation column 350 can advantageously be performed at least partly, ideally completely, by means of the dividing wall column (not shown), as known, for example, from documents EP 012 62 88 B1 or EP 012 23 67 A2.

Overall, a large, energy benefit can be achieved with the plant according to the invention and the process, wherein a significant reduction in the externally supplied energy flows was enabled, in particular saving of large amounts of (external) heating steam.

1. A plant for continuous catalytic hydrogenation of methylenedianiline (MDA; reactant1) with a hydrogen donor (reactant2),
comprising a conditioning unit for the reactants, a reactor unit and a separation unit, wherein
the conditioning unit comprises (feed) conduits for reactant1, reactant2 and at least one solvent, at least one heat exchanger in at least one (feed) conduit, at least one mixer for mixing the reactants and/or at least one reactant with at least one solvent;
the reactor unit comprises at least one fixed bed reactor as main reactor with an immobile catalyst packing, wherein the at least one (first) main reactor comprises a first flow pathway for the mixture of matter through the immobile catalyst packing and another separate closed flow pathway for a heat exchange medium outside the catalyst packing, and
wherein the media circuit incorporates a heat exchanger;
the separation unit comprises at least a first separation stage for essentially removal of the solvent and a second separation stage for separation of the at least one reactant and/or at least one by-product from the product,
wherein
i) a collection circuit for an open or a closed first media circuit is included, in which at least one heat source incorporated is a heat exchanger of the reactor unit, and/or at least one heat exchanger of the separation unit and an evaporator or a distributor tank,
ii) an intermediate circuit for a second media circuit is included, incorporating the first evaporator, at least one fan and a distributor tank, where a (media) conduit leads from the pressure side of the at least one compressor to the distributor tank and a (bottom) outlet of the distributor tank is connected to an inlet of the evaporator via a return conduit, and where
iii) at least one distributor circuit incorporating the distributor tank is included, where the distributor circuit is connected by at least one incoming conduit branch to a tops/vapour outlet of the distributor tank, where the distributor circuit comprises at least one distributor conduit branch and at least one return conduit branch, where a heat sink incorporated into the at least one distributor conduit branch so as to exchange heat is at least one heat exchanger of the conditioning unit, the separation unit, and/or the reaction unit, and where
the intermediate circuit and the distributor circuit are connected to one another via the distributor tank so as to conduct media.

2. The plant according to claim 1, wherein the at least one incoming conduit branch of the distributor circuit incorporates at least one compressor.

3. The plant according to claim 1, wherein the distributor circuit comprises at least the following two (part-) circuits:
at least one high-pressure distributor circuit (HP distributor circuit) connected to a tops/vapour outlet of the distributor tank and incorporating at least one compressor, and at least one low-pressure distributor circuit (LP distributor circuit) connected to a tops/vapour outlet of the distributor tank and incorporating no compressor, fewer compressors and/or a lower compressor output than the HP distributor circuit, and where the HP distributor circuit and the LP distributor circuit incorporate, so as to exchange heat,
as a heat sink at least one heat exchanger of the conditioning unit, separation unit, and/or of the reaction unit, and where the intermediate circuit and the distributor circuit are connected to one another via the distributor tank so as to conduct media.

4. The plant according to claim 1, wherein the intermediate circuit comprises at least one fan or a group of x fans where x is >=2.

5. The plant according to claim 1, wherein the media circuit of the collection circuit incorporates a collection tank and a pump, where a heat exchanger is disposed upstream of the collection tank in the return conduit branch and/or on the suction side of the pump in the incoming conduit branch.

6. The plant according to claim 3, wherein the HP distributor circuit of the distributor circuit comprises a first conduit branch as a vapour-conducting feed, where the feed conduit branch incorporates at least one compressor.

7. The plant according to claim 3, wherein a (bottoms) conduit leads from the distributor tank to the pressure side of the at least one compressor, where this (bottoms) conduit incorporates a pump.

8. The plant according to claim 7, wherein the bottoms conduit from the distributor tank is guided into the (media) conduit between two compressors.

9. The plant according to claim 7, wherein the bottoms conduit has at least two (conduit) branches, where each (conduit) branch of the bottoms conduit is guided between two of the compressors, and where at least one (conduit) branch of the bottoms conduit comprises a pressure control unit.

10. The plant according to claim 3, wherein LP distributor circuit of the distributor circuit comprises at least two part-circuits for distributing or releasing energy, where
the first part-circuit is a low-pressure circuit (LP part-circuit), where the first LP part-circuit incorporates at least one heat exchanger or a first group of two or more heat exchangers, and where
the further part-circuit incorporates at least one heat exchanger or a further group of two or more heat exchangers, with no compressor, fewer compressors and/or a lower compressor output incorporated into the LP part-circuit than in any other part-circuit of the LP distributor circuit.

11. The plant according to claim 10, wherein at least one of the return conduits, returning to the distributor tank, of the distributor circuit, incorporates a heat exchanger.

12. The plant according to claim 1, wherein the heat exchangers of the distributor circuit that act as a heat sink are connected in parallel and/or the respective groups of two or more heat exchangers of the HP distributor circuit and/or the LP distributor circuit are connected in parallel to one other.

13. The plant according to claim 1, wherein the heat exchangers, acting as heat source, of the collection circuit are connected in series.

14. A process for catalytic hydrogenation of methylenedianiline (MDA; reactant1) with a hydrogen donor (reactant2), the process comprises:
implementing a production by an industrial plant,
wherein
the plant is designed according to claim 1, where the main reactor is operated at a temperature in the range from 80° C. to 150° C., and where vapour compression in the intermediate circuit by the at least one fan and/or the fan group results in at least an increase in temperature of the medium in the incoming conduit branch by 20° C. to 60° C.

15. The process according to claim 14, wherein the temperature of the reactant stream at the inlet of the at least one main reactor is 80 to 135° C.

16. The process according to claim 14, wherein the pressure in the main reactor is operated in the range from 60 bar to 120 bar.

17. The process according to claim 14, wherein it is implemented continuously and catalytically for production of methylenebis(cyclohexylamine).

18. The process according to claim 14, wherein in addition to the at least one main reactor, at least one catalyst-containing postreactor is also included, where the at least one main reactor and the at least one postreactor are operated at the same or essentially at the same pressure.

19. The process according to claim 14, wherein the energy introduced into the first evaporator by the collection circuit by the intermediate circuit and the at least one incorporated fan and/or the group of x fans is raised
by at least a factor of 1.1 to 2.5 and/or
the output temperature of the tank is raised by 10° C. to 80° C.

20. The process according to claim 14, wherein a multistage pressure elevation in the incoming conduit is effected in the first conduit portion (portion A) in the HP distributor circuit, where, downstream of the distributor tank and
upstream of the first compressor in the conduit, an (input) pressure is 1.5 bar to 5 bar, and a temperature is 100° C. to 150° C. and/or
downstream of the last compressor and upstream of the first heat exchanger acting as a heat sink, a (final) pressure in the conduit is 10 to 30 bar and a temperature is 180° C. to 300° C.
